SEQUENCE LISTING (36 sequences)

SEQ ID NO: 1 - protein, 344 aa, Unknown
  NTM polypeptide
MGVCGYLFLPWKCLVVVSLRLLFLVPTGVPVRSGDATFPKAMDNVTVRQGESATLRCTIDNRVTRVAWLNRSTILYAGNDKWCLDPRVVLLSNTQTQYSIEIQNVDVYDEGPYTCSVQTDNHPKTSRVHLIVQVSPKIVEISSDISINEGNNISLTCIATGRPEPTVTWRHISPKAVGFVSEDEYLEIQGITREQSGDYECSASNDVAAPVVRRVKVTVNYPPYISEAKGTGVPVGQKGTLQCEASAVPSAEFQWYKDDKRLIEGKKGVKVENRPFLSKLIFFNVSEHDYGNYTCVASNKLGHTNASIMLFGPGAVSEVSNGTSRRAGCVWLLPLLVLHLLLKF

SEQ ID NO: 2 - DNA, 1035 bp, Unknown
  Sequence coding NTM polypeptide
atgggggtctgtgggtacctgttcctgccctggaagtgcctcgtggtcgtgtctctcaggctgctgttccttgtacccacaggagtgcccgtgcgcagcggagatgccaccttccccaaagctatggacaacgtgacggtccggcagggggagagcgccaccctcaggtgcactattgacaaccgggtcacccgggtggcctggctaaaccgcagcaccatcctctatgctgggaatgacaagtggtgcctggatcctcgcgtggtccttctgagcaacacccaaacgcagtacagcatcgagatccagaacgtggatgtgtatgacgagggcccttacacctgctcggtgcagacagacaaccacccaaagacctctagggtccacctcattgtgcaagtatctcccaaaattgtagagatttcttcagatatctccattaatgaagggaacaatattagcctcacctgcatagcaactggtagaccagagcctacggttacttggagacacatctctcccaaagcggttggctttgtgagtgaagacgaatacttggaaattcagggcatcaccagggagcagtcaggggactacgagtgcagtgcctccaatgacgtggccgcgcccgtggtacggagagtaaaggtcaccgtgaactatccaccatacatttcagaagccaagggtacaggtgtccccgtgggacaaaaggggacactgcagtgtgaagcctcagcagtcccctcagcagaattccagtggtacaaggatgacaaaagactgattgaaggaaagaaaggggtgaaagtggaaaacagacctttcctctcaaaactcatcttcttcaatgtctctgaacatgactatgggaactacacttgcgtggcctccaacaagctgggccacaccaatgccagcatcatgctatttggtccaggcgccgtcagcgaggtgagcaacggcacgtcgaggagggcaggctgcgtctggctgctgcctcttctggtcttgcacctgcttctcaaattttga

SEQ ID NO: 3 - protein, 973 aa, Unknown [sequence omitted]

SEQ ID NO: 4 - DNA, 5356 bp, Unknown
  Sequence coding PAM polypeptide
gttctgaatgatgactgacgcgggtttgggtgatacccctcacagcccctgtcattccggagtcataaggcacccgcgcgtctagccccagcgccagggcacgcgagcggcgctggagggaggaaagcttccgcctgcgggccggacaaaagtcccgcctgcccacggctttttgcccgccgctcgtgaccgagacgcctcgccgcggccagctcgctgctctcgctggcggatggtgtgtggccgccgcaggacgcccgccgtgcccgggccatgaagtagcggctgctggcggcgccgctgcccaaccgccagccccagccccgcgctgcgctgcccggtcctctcccggcggggtcgtatcggcgtggacatggctggccgcgtccctagcctgctagttctccttgtttttccaagcagctgtttggctttccgaagcccactttctgtctttaagaggtttaaagaaactaccagaccattttccaatgaatgtcttggtaccaccagacccgtagttcctattgattcatcagattttgcattggatattcgcatgcctggggttacacctaaacagtccgatacatacttctgcatgtctatgcgaataccagtggatgaggaagccttcgtgattgacttcaagcctcgagccagcatggatactgtccatcacatgttactttttggatgcaatatgccttcatccactggaagttactggttttgtgatgaaggaacctgtacagataaagccaatattctgtatgcctgggcgagaaatgctccccctacccggctccccaaaggtgttggattcagagttggaggagagactggaagtaaatactttgtactacaggtacactatggggatattagtgcttttagagataataacaaggactgttctggtgtgtccttacacctcacacgtctgccacagcctttaattgctggcatgtaccttatgatgtctgttgacactgttatcccagcaggagaaaaagtggtgaattctgacatttcatgccattataaaaattatccaatgcatgtctttgcctatagagttcacactcaccatttaggtaaggtagtaagtggatacagagtaagaaatggacagtggacactgattggacggcagagccctcagctgccacaggctttctaccctgtggggcatccagttgatgtaagttttggtgacctactggctgcaagatgtgtattcactggtgaaggaaggacagaagccacacacattggtggcacgtctagtgatgaaatgtgcaacttatacattatgtattacatggaagccaagcatgcagtttctttcatgacctgtacccagaatgtagctccagatatgttcagaaccataccaccagaggccaacattccaattcccgtgaagtctgatatggttatgatgcatgaacatcataaagaaacagaatataaagataagattcctttactacagcagccaaaacgagaagaagaagaagtgttagaccagggtgatttctattcactactttccaagctgctaggagaaagggaagatgttgttcatgtgcacaaatataatcctacagaaaaggcagaatcagagtcagacctggtagctgagattgcaaatgtagtccaaaaaaaggatcttggtcgatctgatgccagagagggtgcagaacatgagaggggtaatgctattcttgtcagagacagaattcacaaattccacagactagtatctaccttgaggccaccagagagcagagttttctcattacagcagcccccacctggtgaaggcacctgggaaccagaacacacaggagatttccacatggaagaggcactggattggcctggagtatacttgttaccaggccaggtttctggggtggctctagaccctaagaataacctggtgattttccacagaggtgaccatgtctgggatggaaactcgtttgacagcaagtttgtttaccagcaaataggactcggaccaattgaagaagacactattcttgtcatagatccaaataatgctgcagtactccagtccagtggaaaaaatctgttttacttgccacatggcttgagtatagataaagatgggaattattgggtcacagacgtggctctccatcaggtgttcaaactggatccaaacaataaagaaggccctgtattaatcctgggaaggagcatgcaaccaggcagtgaccagaatcacttctgtcaacccactgatgtggctgtggatccaggcactggagccatttatgtatcagatggttactgcaacagcaggattgtgcagttttcaccaagtggaaagttcatcacacagtggggagaagagtcttcagggagcagtcctctgccaggccagttcactgttcctcacagcttggctcttgtgcctcttttgggccaattatgtgtggcagaccgggaaaatggtcggatccagtgttttaaaactgacaccaaagaatttgtgagagagattaagcattcatcatttggaagaaatgtatttgcaatttcatatataccaggcttgctctttgcagtgaatgggaagcctcattttggggaccaagaacctgtacaaggatttgtgatgaacttttccaatggggaaattatagacatcttcaagccagtgcgcaagcactttgatatgcctcatgatattgttgcatctgaagatgggactgtgtacattggagatgctcataccaacaccgtgtggaagttcaccttgactgagaaattggaacatcgatcagttaaaaaggctggcattgaggtccaggaaatcaaagaagccgaggcagttgttgaaaccaaaatggagaacaaacccacctcctcagaattgcagaagatgcaagagaaacagaaactgatcaaagagccaggctcgggagtgcctgttgttctcattacaacccttctggttattccggtggttgtcctgctggccattgccatatttattcggtggaaaaaatcaagggcctttggagattctgaacacaaactcgagacgagttcaggaagagtactgggaagatttagaggaaagggaagtggaggcttaaaccttggtaatttctttgcaagccgtaagggctacagtcgaaaagggtttgaccggcttagcactgagggcagtgaccaagagaaagaggatgatggaagtgaatcagaagaggagtattcagcacctctgcctgcgctcgcaccttcctcctcctgaaaaccaagctttgatttagattgagtaagatttacccagaatgtcagattcctttccctttagcacgtttaaagttctgtgtatttaattgtaaactgtactagtctgtgtgggactgtacacactttatttacttcgttttggttaagttggcttctgtttctagttgaggagtttcctaaaagttcataacagtgccattgtctttatatgaacatagactagagaaaccgtcctctttttccatcataattctaatctaacaatggaagatttgcccatttacacttttgagactttttggtggatgtaaataaccccattctttgcttgaacacagtattttcccaatagcactttcattgccagtgtctttctttggtgcctttcctgttcagcattcttagcctgtggcagtaaagagaaactttgtgctacatgacgacaaagctgctaaatctcctatttttttaaaatcactaacattatattgcaatgaaggaaataaaaaagtctctatttaaattcttttttaaattttcttcagttggtgtgtttttgggatgtcttatttttagatggttacactgttagaacactattttcagaatctgaatgtaatttgtgtaataaagtgttttcagagcattagctgtcagtgtattttccagtttttgcgtatttgcagattttacatacaacttttataataattacacaaacccacaaatattagtgaaacttactcgatgtcttcaactaaaagaaatgtgtgtattgtacaaaatttagaagatactttagccaatataaattaaaaaccagcctgagtttacataaatttgtaaagtcaggctcttctaaaatccaaagagggtttttgcctatatatcaatcagaggataaatactttaataaaaggtaatcacagctaagtggatacctgtgtctcaaattacatatgcaaatgatccatcagtagggatcactaataatagttttccttttaaaaaataatttcagggcaggtacagtggctcaagcctatacttccaggactttgggaggccaagcagaaggattgtgggaggccaagcagaaggattgtttgagcccagaaattcaaggctgcagtgagctatgatcaatccactgtactccagcctaggctacagagtgagatcctttctctaaaataaaatacaatttccatgtatccataggaatatattcatcttttacagtgattgcagattagttttaaaacgtctttttcgtaattcgtcaatgcaagttagtaagaaccagataattttccattttaaaatgataggaatctaaaactctttttcaaaaatgccaacctgtttttccggcatcatagtagttggaataatacagatataattgactaatcataaagtacatgagagtacagaagggaaatttggaaacggtaagtctgctagggcattacaatcctgtcctagcactccacctttattctgccaacctgggttaattaaagatggtagcctggaaagtaatgaatgacattgacttcaggcaatctttccactgattattcttggctgaacttcatttatcgagtcagagaatgcactgcctgagaaatgtcccagaggagtgaatcctaggcctggccacagtagaatcgcctggagaatttaagaaaaaaattgttgggcactcactctttccagtgatctttatttagttggcttacaatggaatacaggtatggttcatgtttttaaatttgtccaggtgtttctattgtgcagtcacagttgaaaaccattgtcttcgagaatagctattctatcttgccagttacaataagtggatagctatattttcacataaattatagtttacagatgtttggagggggaagacaggatctgaggtgttttgatatgactgttagcaccaaaatctgaatgccttaattgttgaatgtgttaaattggataattaaaatgggcataaatgacttattaaaaaagcaaagggaaaaaaaaaaaaaaaaaaaaaaaa

SEQ ID NO: 5 - protein, 1015 aa, Unknown
  PTPRN2 polypeptide
MGPPLPLLLLLLLLLPPRVLPAAPSSVPRGRQLPGRLGCLLEEGLCGASEACVNDGVFGRCQKVPAMDFYRYEVSPVALQRLRVALQKLSGTGFTWQDDYTQYVMDQELADLPKTYLRRPEASSPARPSKHSVGSERRYSREGGAALANALRRHLPFLEALSQAPASDVLARTHTAQDRPPAEGDDRFSESILTYVAHTSALTYPPGSRTQLREDLLPRTLGQLQPDELSPKVDSGVDRHHLMAALSAYAAQRPPAPPGEGSLEPQYLLRAPSRMPRPLLAPAAPQKWPSPLGDSEDPSSTGDGARIHTLLKDLQRQPAEVRGLSGLELDGMAELMAGLMQGVDHGVARGSPGRAALGESGEQADGPKATLRGDSFPDDGVQDDDDRLYQEVHRLSATLGGLLQDHGSRLLPGALPFARPLDMERKKSEHPESSLSSEEETAGVENVKSQTYSKDLLGQQPHSEPGAAAFGELQNQMPGPSKEEQSLPAGAQEALSDGLQLEVQPSEEEARGYIVTDRDPLRPEEGRRLVEDVARLLQVPSSAFADVEVLGPAVTFKVSANVQNVTTEDVEKATVDNKDKLEETSGLKILQTGVGSKSKLKFLPPQAEQEDSTKFIALTLVSLACILGVLLASGLIYCLRHSSQHRLKEKLSGLGGDPGADATAAYQELCRQRMATRPPDRPEGPHTSRISSVSSQFSDGPIPSPSARSSASSWSEEPVQSNMDISTGHMILSYMEDHLKNKNRLEKEWEALCAYQAEPNSSFVAQREENVPKNRSLAVLTYDHSRVLLKAENSHSHSDYINASPIMDHDPRNPAYIATQGPLPATVADFWQMVWESGCVVIVMLTPLAENGVRQCYHYWPDEGSNLYHIYEVNLVSEHIWCEDFLVRSFYLKNLQTNETRTVTQFHFLSWYDRGVPSSSRSLLDFRRKVNKCYRGRSCPIIVHCSDGAGRSGTYVLIDMVLNKMAKGAKEIDIAATLEHLRDQRPGMVQTKEQFEFALTAVAEEVNAILKALPQ

SEQ ID NO: 6 - DNA, 4859 bp, Unknown
  Sequence conding PTPRN2 polypeptide
cggcgtcgccgcaagagcgcgagaggcgcggcgggaggagccggcggcgcgcgcctccctgggcccggactcggccgcctcccgccgcctcccgcgcggccatggactgagcgccgccggccaggccgcggggatggggccgccgctcccgctgctgctgctgctactgctgctgctgccgccacgcgtcctgcctgccgccccttcgtccgtcccccgcggccggcagctcccggggcgtctgggctgcctgctcgaggagggcctctgcggagcgtccgaggcctgtgtgaacgatggagtgtttggaaggtgccagaaggttccggcaatggacttttaccgctacgaggtgtcgcccgtggccctgcagcgcctgcgcgtggcgttgcagaagctttccggcacaggtttcacgtggcaggatgactatactcagtatgtgatggaccaggaacttgcagacctcccgaaaacctacctgaggcgtcctgaagcatccagcccagccaggccctcaaaacacagcgttggcagcgagaggaggtacagtcgggagggcggtgctgccctggccaacgccctccgacgccacctgcccttcctggaggccctgtcccaggccccagcctcagacgtgctcgccaggacccatacggcgcaggacagaccccccgctgagggtgatgaccgcttctccgagagcatcctgacctatgtggcccacacgtctgcgctgacctaccctcccgggtcccggacccagctccgcgaggacctcctgccgcggaccctcggccagctccagccagatgagctcagccctaaggtggacagtggtgtggacagacaccatctgatggcggccctcagtgcctatgctgcccagaggcccccagctccccccggggagggcagcctggagccacagtaccttctgcgtgcaccctcaagaatgcccaggcctttgctggcaccagccgccccccagaagtggccttcacctctgggagattccgaagacccctccagcacaggcgatggagcacggattcataccctcctgaaggacctgcagaggcagccggctgaggtgaggggcctgagtggcctggagctggacggcatggctgagctgatggctggcctgatgcaaggcgtggaccatggagtagctcgaggcagccctgggagagcggccctgggagagtctggagaacaggcggatggccccaaggccaccctccgtggagacagctttccagatgacggagtgcaggacgacgatgatagactttaccaagaggtccatcgtctgagtgccacactcgggggcctcctgcaggaccacgggtctcgactcttacctggagccctcccctttgcaaggcccctcgacatggagaggaagaagtccgagcaccctgagtcttccctgtcttcagaagaggagactgccggagtggagaacgtcaagagccagacgtattccaaagatctgctggggcagcagccgcattcggagcccggggccgctgcgtttggggagctccaaaaccagatgcctgggccctcgaaggaggagcagagccttccagcgggtgctcaggaggccctcagcgacggcctgcaattggaggtccagccttccgaggaagaggcgcggggctacatcgtgacagacagagaccccctgcgccccgaggaaggaaggcggctggtggaggacgtcgcccgcctcctgcaggtgcccagcagtgcgttcgctgacgtggaggttctcggaccagcagtgaccttcaaagtgagcgccaatgtccaaaacgtgaccactgaggatgtggagaaggccacagttgacaacaaagacaaactggaggaaacctctggactgaaaattcttcaaaccggagtcgggtcgaaaagcaaactcaagttcctgcctcctcaggcggagcaagaagactccaccaagttcatcgcgctcaccctggtctccctcgcctgcatcctgggcgtcctcctggcctctggcctcatctactgcctccgccatagctctcagcacaggctgaaggagaagctctcgggactagggggcgacccaggtgcagatgccactgccgcctaccaggagctgtgccgccagcgtatggccacgcggccaccagaccgacctgagggcccgcacacgtcacgcatcagcagcgtctcatcccagttcagcgacgggccgatccccagcccctccgcacgcagcagcgcctcatcctggtccgaggagcctgtgcagtccaacatggacatctccaccggccacatgatcctgtcctacatggaggaccacctgaagaacaagaaccggctggagaaggagtgggaagcgctgtgcgcctaccaggcggagcccaacagctcgttcgtggcccagagggaggagaacgtgcccaagaaccgctccctggccgtgctgacctatgaccactcccgggtcctgctgaaggcggagaacagccacagccactcagactacatcaacgctagccccatcatggatcacgacccgaggaaccccgcgtacatcgccacccagggaccgctgcccgccaccgtggctgacttttggcagatggtgtgggagagcggctgcgtggtgatcgtcatgctgacacccctcgcggagaacggcgtccggcagtgctaccactactggccggatgaaggctccaatctctaccacatctatgaggtgaacctggtctccgagcacatctggtgtgaggacttcctggtgaggagcttctatctgaagaacctgcagaccaacgagacgcgcaccgtgacgcagttccacttcctgagttggtatgaccgaggagtcccttcctcctcaaggtccctcctggacttccgcagaaaagtaaacaagtgctacaggggccgttcttgtccaataattgttcattgcagtgacggtgcaggccggagcggcacctacgtcctgatcgacatggttctcaacaagatggccaaaggtgctaaagagattgatatcgcagcgaccctggagcacttgagggaccagagacccggcatggtccagacgaaggagcagtttgagttcgcgctgacagccgtggctgaggaggtgaacgccatcctcaaggcccttccccagtgagcggcagcctcaggggcctcaggggagcccccaccccacggatgttgtcaggaatcatgatctgactttaattgtgtgtcttctattataactgcatagtaatagggcccttagctctcccgtagtcagcgcagtttagcagttaaaagtgtatttttgtttaatcaaacaataataaagagagatttgtggaaaaatccagttacgggtggaggggaatcggttcatcaattttcacttgcttaaaaaaaatactttttcttaaagcacccgttcaccttcttggttgaagttgtgttaacaatgcagtagccagcacgttcgaggcggtttccaggaagagtgtgcttgtcatctgccactttcgggagggtggatccactgtgcaggagtggccggggaagctggcagcactcagtgaggccgcccggcacacaaggcacgtttggcatttctctttgagagagtttatcattgggagaagccgcggggacagaactgaacgtcctgcagcttcggggcaagtgagacaatcacagctcctcgctgcgtctccatcaacactgcgccgggtaccatggacggccccgtcagccacacctgtcagcccaagcagagtgattcaggggctccccgggggcagacacctgtgcaccccatgagtagtgcccacttgaggctggcactcccctgacctcacctttgcaaagttacagatgcaccccaacattgagatgtgtttttaatgttaaaatattgatttctacgttatgaaaacagatgcccccgtgaatgcttacctgtgagataaccacaaccaggaagaacaaatctgggcattgagcaagctatgagggtccccgggagcacacgaaccctgccaggcccccgctggctcctccaggcacgtcccggacctgtggggccccagagaggggacatttccctcctgggagagaaggagatcagggcaactcggagagggctgcgagcatttccctcccgggagaggagatcagggcgacctgcacgcactgcgtagagcctggaagggaagtgagaaaccagccgaccggccctgcccctcttcccgggatcacttaatgaaccacgtgttttgacatcatgtaaacctaagcacgtagagatgattcggatttgacaaaataacatttgagtatccgattcgccatcaccccctaccccagaaataggacaattcacttcattgaccaagatgatcacatggaaggcggcacagaggcagctgtgtgggctgcagatttcctgtgtggggttcagcgtagaaaacgcacctccatcccgcccttcccacagcattcctccatcttagatagatggtactctccaaaggccctaccagagggaacacggcctactgagcggacagaatgatgccaaaatattgcttatgtctctacatggtattgtaatgaatatctgctttaatatagctatcatttcttttccaaaattacttctctctatctggaatttaattaatcgaaatgaatttatctgaatataggaagcatatgcctacttgtaatttctaactccttatgtttgaagagaaacctccggtgtgagatatacaaatatatttaattgtgtcatattaaacttctgatttcacattcaaaaaaaaaaaaaaaaa

SEQ ID NO: 7 - protein, 140 aa, Unknown
  LY6H polypeptide
MLPAAMKGLGLALLAVLLCSAPAHGLWCQDCTLTTNSSHCTPKQCQPSDTVCASVRITDPSSSRKDHSVNKMCASSCDFVKRHFFSDYLMGFINSGILKVDVDCCEKDLCNGAAGAGHSPWALAGGLLLSLGPALLWAGP

SEQ ID NO: 8 - DNA, 950 bp, Unknown
  Sequence coding LY6H polypeptide
agtggcaggtcccacccgctcccgcccctgccgctcattcctgtccggacgcacctgcagctccccggggagaagaggacgccccccatccgctcccccacacaggcttgcgccccagaggacccgcgccccaagcccccgcgccgcccccaggcccacccggagcatgctgcctgcagccatgaagggcctcggcctggcgctgctggccgtcctgctgtgctcggcgcccgctcatggcctgtggtgccaggactgcaccctgaccaccaactccagccattgcaccccaaagcagtgccagccgtccgacacggtgtgtgccagtgtccgaatcaccgatcccagcagcagcaggaaggatcactcggtgaacaagatgtgtgcctcctcctgtgacttcgttaagcgacactttttctcagactatctgatggggtttattaactctgggatcttaaaggtcgacgtggactgctgcgagaaggatttgtgcaatggggcggcaggggcagggcacagcccctgggccctggccggggggctcctgctcagcctggggcctgccctcctctgggctgggccctgatgtctcctccttcccacggggcttctgagcttgctcccctgagcctgtggctgccctctccccagcctggcgtggctggggctgggggcagccttggcccagctccgtggctgtggcctgtggctctcactcctcccccgacgtgaagcctccctgtctctccgccagctctgagtcccaggcagctggacatctccaggaaaccaggccatctgggcaggaggcctggggatgagggtggggggggacccccaggtcccggaggggaagtgaagcaacagcccagctggaagggcgtcttctgcggagaaataaagtcacttttgagtcctgagaaaaaaaaaaaaaaaaaaaaaaaaaa

SEQ ID NO: 9 - protein, 345 aa, Unknown
  OPCML amino acid
MGVCGYLFLPWKCLVVVSLRLLFLVPTGVPVRSGDATFPKAMDNVTVRQGESATLRCTIDDRVTRVAWLNRSTILYAGNDKWSIDPRVIILVNTPTQYSIMIQNVDVYDEGPYTCSVQTDNHPKTSRVHLIVQVPPQIMNISSDITVNEGSSVTLLCLAIGRPEPTVTWRHLSVKEGQGFVSEDEYLEISDIKRDQSGEYECSALNDVAAPDVRKVKITVNYPPYISKAKNTGVSVGQKGILSCEASAVPMAEFQWFKEETRLATGLDGMRIENKGRMSTLTFFNVSEKDYGNYTCVATNKLGNTNASITLYGPGAVIDGVNSASRALACLWLSGTLLAHFFIKF

SEQ ID NO: 10 - DNA, 6748 bp, Unknown
  Sequence conding OPCML polypeptide
tccttccgtcgctcaggagagtggagccaatgcgtgccttggaggactgctctgagccctgtgcagattggtgctgatgattcttaccagttgtgtgcaagaaaagaaatcagagacgctgcctgcctgctcccatctctcgcgcgctctctctctcttctgctctctccctccctttgcaaacattggatttaaacctgctcagaattcagcacagaggaaggcagcagcggtagcagcagcagaagcagtagcaagcccggcagctgagagcaccgcagcgtcgagatgtaccatcctgcctactgggtcgtcttctcggcgacaactgccctgctcttcatcccaggtgtaccatagatgaccgggtaacccgggtggcctggctaaaccgcagcaccatcctctacgctgggaatgacaagtggtccatagaccctcgtgtgatcatcctggtcaatacaccaacccagtacagcatcatgatccaaaatgtggatgtgtatgacgaaggtccgtacacctgctctgtgcagacagacaatcatcccaaaacgtcccgggttcacctaatagtgcaagttcctcctcagatcatgaatatctcctcagacatcactgtgaatgagggaagcagtgtgaccctgctgtgtcttgctattggcagaccagagccaactgtgacatggagacacctgtcagtcaaggaaggccagggctttgtaagtgaggatgagtacctggagatctctgacatcaagcgagaccagtccggggagtacgaatgcagcgcgttgaacgatgtcgctgcgcccgatgtgcggaaagtaaaaatcactgtaaactatcctccctatatctcaaaagccaagaacactggtgtttcagtcggtcagaagggcatcctgagctgtgaagcctctgcagtccccatggctgaattccagtggttcaaggaagaaaccaggttagccactggtctggatggaatgaggattgaaaacaaaggccgcatgtccactctgactttcttcaatgtttcagaaaaggattatgggaactatacttgtgtggccacgaacaagcttgggaacaccaatgccagcatcacattgtatgggcctggagcagtcattgatggtgtaaactcggcctccagagcactggcttgtctctggctatcagggaccctcttagcccacttcttcatcaagttttgataagaaatcctaggtcctctgagcaacgcctgcttctccatatcacagactttaatctacactgcggagagcaaaccagcttgggcttctttttgtttttttctgttattctagatttgttttctttttgtttttgtttatttgtttgtttgcttttatttccagcttgaatgagtggggttgggggcggggtgggcagggttctaccacgtgtaggataatcattcattggtgtgtccaaaaatggggtctgctcctgctaccttgacccttccctttcctctgcttctctcctcatcatcattcccaacaacatcctctgccacacacaacaaaacgtaagtttcatttgggcaaaaattgagcctcacaataaacaccctgaagacacaacttgacttataacatagtgcacagcaagagctacatccaagtgtcctattatctgtgattattttcttaatgacaatgtacatatgcccccatccatgttaattattatctaattccattagggttcacgtcttttctttctgggacactatcctactatatccatatctatagatttcaatatagatgattgtgccatcttctgtagcccctccgctctactcattccttccaccatctgcagagatttgaagtttggggctatgcatgaaacccaacactaaattttgcaagtcaagtaaccaaaaaagggggaggcattttgaagatagaacctctattttaaaaagagaagttcaactcataaacgtgattgataggtggctgatttatttaggttttgtcaagctatctatcaaagtaatggtacagttacccatctactcaaatatctgatttatctcaccatccaattatctacccacctgtcttcctctctagcaatctatttactgtttatcaatctatcaatgtaattgtctaacactcctttctattctctccctactactcactatcaattcatccccatatgaatctctaaccatattgtatctctcccactgtattcatttatacaccatcagcagacattggcatcttcaaaattatctttcaacttctgtgaaagccaacgatctcacaggttaacaaaatacaaaagcaataccctgtgttgtggactctttaaaatctggtatcctatccacccaagggagacactaacagataggccaaagtagcaagctaatgatcagtcactcactattcccagaagagcctgtgttttctaaaacactttcttgggaagcagatcagcctagaaaagttttgattagcactgtggttttccttttgcacttgaaggacaaaggtgccagcctttatgcttctctcaacccttcaagaaagtacatgtcaggaacctatggctggctttccttagcagcaagaacttgagagaaaaacacatctgtctctgcaatgcaaagtgaagagtccacccgcctgagtgggatgacttcagctagagtctcctttctgctccagttctggtttaatctgtttgaaaactatccagtaaaaagctgatggaggccaattacatggcgggtgtattgacaactctggtatttgtttcaggaagctcttctaagctgagggcacttgagcaactgacttaattttcaagcacttgattaacacaacactgcaaacagaagggagaaagtgtcagtgacacagtttcctctgatgcagctgcttctccaatggctttggggaagaacttcaccagctcttcaggttcaaagcagacccagcatacaaacaagagctgagccacctttgctgtcttgtctcctgggacgagaaggactcatccagcaaagttgcctgggattcaaaataaaggcattgcagaccgcacaggtgtgctgcagggactgatccacagagaggatgagaatgcagcatcaatcgcagacctgccctgcctcagttggaaaaccttttcaggccctcagtctaaaaaataaaaaatatgagcaccattgaattctgtgcccttaatgcttaactggtcttcttcctctggtatcagtgtcctctttgtttttgtccatcaaggcacatgagtgtgacctctgccatggggaaacacacacagagatatctatacatatatacatacatacaaacataggctatcttggcacactaaatgctaagcactgtcttaagaggtagagctggtgtgagtgaaattaatgttacattttccagctgtaaacagacatctgcatttcctagtgagctgccaggagccagattcgggaaccgtaactgatgtgccaggaatggtgcattgattcccagttccagggatgatcatgagcaggcgcaaaatcagaattaaaggtcgcacatagacgtttcagatctgtcaccaccttcagcatctggagttgagttggtgtcagatagtgtatgagaattaaatgtgtcatctgagcatgctactgatgataaatttgttactttggagttgaataaatgtgaaggctgtgaagagtggacagtcttggagaacacagtgcttgaaatggacaagctggacctattcctcactccaagacttgttctacaggaaagggtccatgctcctttggccaagatcatcagaacctctcaacccaacaaggctggcttcagggccactatggaaccctgctgttcccccttccaaaggatactaagatgcccctctggtgggtacctatcccagccacgtttcagagggagagaaatgctacagttgatcctcatctgtctggggtaaagacaacaaagtaaatacaacccaaggcaactggggtactcactgggagtgaaaatgacttcttcacaacagacatatttctgcttctgtgtttttgtgtttctttggtggggatggcttcatgggagagtggctgtcacccatcattttgaagcatatagaacaacaaatgcttacacaagacaatatccacacttttccaacttcacacacggagagtacatggagaatgcctacaggctagatttgttcagggtgccagtagtgggcatggggtgggggcaaggcaggacaaaacatacaagtctgagcaagtacatctcttgcaggttttccacatgaaaaggaagccaaataagtcctgttaggagattaggtgagaggaattagcaatgtagggactctgaaacccttccccttcccaaaacagagttcatatgcacttccaccaaagtaatgccaatgaaagtgctcgtgttaaggctgcagccaagcttgtttttcagtagtttaatgtcaagtgcctgatacagtcgactgcaagtctaaacaagcatgtttagtttttctcattcttgctttaattcagggagggggagatgtagagaagtggttgtgaaaacatgtacaggctttatgcagagcactgcgcatggctgttctgctgcaactgtgctccacgaaacagaagaaaaggtaaggtgttgtgtcacaaagaggccccagtctctttcttcttacatccatgcctcttactagatgatacatttacagattgggcagtttgttctcaaaacctgggtgagaagactattcctggactctagcaacttcaaaactgaggctgggtttcagaatctttttctgcatcaattcagtcaatttgccttcaacaaagagaagtcagcaagttctatttatgctgaaagaactattccatgagaaaagcagagaaccccaaagtgggcaggcaaccccgacgagagcttatccctgtggcggcatcaggagtggctgtacattgaattttcaagtgctggttggctgtcgccagcccatggtaggaggggaggaatggcttaagatgaggtaagatctggtggtggggcatctttcctcaattccatactgactttgatcttgagaaagaaaaactggctatgcattacctaaaaccagtccaaaatgaaacagaccaacacacacacaaaagcaaattgtcaatccctttggaattaagggaagcagcataaggtttttctttttggaaaaaatgcatttattttctttttctccaacagcaagaatcttttgttttcattttgcacgtgaccttatcttggaaactcttatacccaattgcctcccctcctattattcagagcttccctgtctttttacttgaagacaaataagtttgagcacttgagtaaaacttcacaggtgtgtaagtaggaaggcaacattttcaaaaagagaccatatgatgagaacgcctaatgatcaccacatgcaaacaaacaaaactgccagtctcatttcccacatttcttacttaagagaagagaagtaaatgaaaggaagaagaaatagatttgtaattaaagatgtggcaaaaaagatagggctgagccagttcaatttagccttcaggtgcagaatacttagagtccaaagaaatgtggagtggacttaattagatgcagttgtctttatcctgaaagtagtgagctaagcctaatttccagcattttgaaagagattcctttttgtttctttccatggtgccctctttaaggcacagagttgctccacaccactgggtggagaaagaaagattgcgaaccctcgaccatccttttgaggctacattctatgttatttggcagatttataaagctatcagtaataacaatgctatgtactgcaagctgcccttgtgttagttaaagggagcatttttaatcgttcggaaattttcgtgacatgtcaagtgcagttgtgaggactgtgtgggtgaacgaaaatgtgtctgtcaagttcagagtcctttagatttaaaaaaaaattatgacttatcaatggtgccgttatagctgtgtcagacaatgggtgtgcccattctcacaattatccttcaaaaaaaatctatgttcaaatgctttaaaaatttatcacacgatacaagagtatgactttgtcagccttctagagttctttttttcttttattttctttcgtattttttccttcaaaaaatcaatgaagacttgatttctgtcaataattgtatcaagggtgaatatactacctgaattttgtgcatgttacattgtagttgtaaccttttctaattcaggatgaatacgagatggttgtgattgtgcagtgtaccaataaagttcgagaaatttgtaacttttggggggctgtttattaggacatctcattgatatccaggacgggaaaaatatgaagcgagctctgcttctcccatatgtgcgtttttggccctggagagtagatgcactcagaaagcctggagctgcaccccccacctctctctctccctccttctctctctcgcacacactgtagaaagcagccttagcactgtccgtatgacatttgtatatcaaagatctatgctgtttacaaacaatataaaatgtagctgcagcctcaaagtctaaattgtttgttggtgtaggcgaaaacgaaagcagaagagaag

SEQ ID NO: 11 - protein, 245 aa, Unknown
  YWHAZ amino acid
MDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQAESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDTQGDEAEAGEGGEN

SEQ ID NO: 12 - DNA, 3020 bp, Unknown
  Sequence conding YWHAZ polypeptide
tgtcccctcgcgcagtcaccgagcgcagatcccgcgctctcgattggaacgcctccccatcactcagccacactcaggggctggacgcctcactcccgtttccgagccataaaaggtctaggaccgcttcccccgagccagcagaacatccagtcatggataaaaatgagctggttcagaaggccaaactggccgagcaggctgagcgatatgatgacatggcagcctgcatgaagtctgtaactgagcaaggagctgaattatccaatgaggagaggaatcttctctcagttgcttataaaaatgttgtaggagcccgtaggtcatcttggagggtcgtctcaagtattgaacaaaagacggaaggtgctgagaaaaaacagcagatggctcgagaatacagagagaaaattgagacggagctaagagatatctgcaatgatgtactgtctcttttggaaaagttcttgatccccaatgcttcacaagcagagagcaaagtcttctatttgaaaatgaaaggagattactaccgttacttggctgaggttgccgctggtgatgacaagaaagggattgtcgatcagtcacaacaagcataccaagaagcttttgaaatcagcaaaaaggaaatgcaaccaacacatcctatcagactgggtctggcccttaacttctctgtgttctattatgagattctgaactccccagagaaagcctgctctcttgcaaagacagcttttgatgaagccattgctgaacttgatacattaagtgaagagtcatacaaagacagcacgctaataatgcaattactgagagacaacttgacattgtggacatcggatacccaaggagacgaagctgaagcaggagaaggaggggaaaattaaccggccttccaacttttgtctgcctcattctaaaatttacacagtagaccatttgtcatccatgctgtcccacaaatagttttttgtttacgatttatgacaggtttatgttacttctatttgaatttctatatttcccatgtggtttttatgtttaatattaggggagtagagccagttaacatttagggagttatctgttttcatcttgaggtggccaatatggggatgtggaatttttatacaagttataagtgtttggcatagtacttttggtacattgtggcttcaaaagggccagtgtaaaactgcttccatgtctaagcaaagaaaactgcctacatactggtttgtcctggcggggaataaaagggatcattggttccagtcacaggtgtagtaattgtgggtactttaaggtttggagcacttacaaggctgtggtagaatcataccccatggataccacatattaaaccatgtatatctgtggaatactcaatgtgtacacctttgactacagctgcagaagtgttcctttagacaaagttgtgacccattttactctggataagggcagaaacggttcacattccattatttgtaaagttacctgctgttagctttcattatttttgctacactcattttatttgtatttaaatgttttaggcaacctaagaacaaatgtaaaagtaaagatgcaggaaaaatgaattgcttggtattcattacttcatgtatatcaagcacagcagtaaaacaaaaacccatgtatttaacttttttttaggatttttgcttttgtgatttttttttttttgatacttgcctaacatgcatgtgctgtaaaaatagttaacagggaaataacttgagatgatggctagctttgtttaatgtcttatgaaattttcatgaacaatccaagcataattgttaagaacacgtgtattaaattcatgtaagtggaataaaagttttatgaatggacttttcaactactttctctacagcttttcatgtaaattagtcttggttctgaaacttctctaaaggaaattgtacattttttgaaatttattccttattccctcttggcagctaatgggctcttaccaagtttaaacacaaaatttatcataacaaaaatactactaatataactactgtttccatgtcccatgatcccctctcttcctccccaccctgaaaaaaatgagttcctattttttctgggagagggggggattgattagaaaaaaatgtagtgtgttccatttaaaattttggcatatggcattttctaacttaggaagccacaatgttcttggcccatcatgacattgggtagcattaactgtaagttttgtgcttccaaatcactttttggtttttaagaatttcttgatactcttatagcctgccttcaattttgatcctttattctttctatttgtcaggtgcacaagattaccttcctgttttagccttctgtcttgtcaccaaccattcttacttggtggccatgtacttggaaaaaggccgcatgatctttctggctccactcagtgtctaaggcaccctgcttcctttgcttgcatcccacagactatttccctcatcctatttactgcagcaaatctctccttagttgatgagactgtgtttatctccctttaaaaccctacctatcctgaatggtctgtcattgtctgcctttaaaatccttcctctttcttcctcctctattctctaaataatgatggggctaagttatacccaaagctcactttacaaaatatttcctcagtactttgcagaaaacaccaaacaaaaatgccattttaaaaaaggtgtattttttcttttagaatgtaagctcctcaagagcagggacaatgttttctgtatgttctattgtgcctagtacactgtaaatgctcaataaatattgatgatgggaggcagtgagtcttgatgataagggtgagaaactgaaatcccaaacactgttttgttgcttgttttattatgacctcagattaaattgggaaatattggcccttttgaataattgtcccaaatattacattcaaataaaagtgcaatggagaaaaaaaaaaa

SEQ ID NO: 13 - protein, 457 aa, Unknown
  CHGA amino acid
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVMEKREDSKEAEKSGEATDGARPQALPEPMQESKAEGNNQAPGEEEEEEEEATNTHPPASLPSQKYPGPQAEGDSEGLSQGLVDREKGLSAEPGWQAKREEEEEEEEEAEAGEEAVPEEEGPTVVLNPHPSLGYKEIRKGESRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQLQALRRG

SEQ ID NO: 14 - DNA, 2079 bp, Unknown
  Sequence coding CHGA polypeptide
gtcatttccggggtcggggtatataagcggggcgcgagggcgctgctgctgccaccgctcctgccactgcagtgctcgagccccgtgcaggggagcttgcgggaggatcgaccgacagacggacgcacgccgaggcactgcgcccccagccccgcgccggtgccaccgcagcccgaccccggccgccagtccagccgcccctcgcccggtgcctaggtgcccggccccacaccgccagctgctcggcgcccgggtccgccatgcgctccgccgctgtcctggctcttctgctctgcgccgggcaagtcactgcgctccctgtgaacagccctatgaataaaggggataccgaggtgatgaaatgcatcgttgaggtcatctccgacacactttccaagcccagccccatgcctgtcagccaggaatgttttgagacactccgaggagatgaacggatcctttccattctgagacatcagaatttactgaaggagctccaagacctcgctctccaaggcgccaaggagagggcacatcagcagaagaaacacagcggttttgaagatgaactctcagaggttcttgagaaccagagcagccaggccgagctgaaagaggcggtggaagagccatcatccaaggatgttatggagaaaagagaggattccaaggaggcagagaaaagtggtgaagccacagacggagccaggccccaggccctcccggagcccatgcaggagtccaaggctgaggggaacaatcaggcccctggggaggaagaggaggaggaggaggaggccaccaacacccaccctccagccagcctccccagccagaaatacccaggcccacaggccgagggggacagtgagggcctctctcagggtctggtggacagagagaagggcctgagtgcagagccagggtggcaggcaaagagagaagaggaggaggaggaggaggaggaggctgaggctggagaggaggctgtccccgaggaagaaggccccactgtagtgctgaacccccacccgagccttggctacaaggagatccggaaaggcgagagtcggtcggaggctctggctgtggatggagctgggaagcctggggctgaggaggctcaggaccccgaagggaagggagaacaggagcactcccagcagaaagaggaggaggaggagatggcagtggtcccgcaaggcctcttccggggtgggaagagcggagagctggagcaggaggaggagcggctctccaaggagtgggaggactccaaacgctggagcaagatggaccagctggccaaggagctgacggctgagaagcggctggaggggcaggaggaggaggaggacaaccgggacagttccatgaagctctccttccgggcccgggcctacggcttcaggggccctgggccgcagctgcgacgaggctggaggccatcctcccgggaggacagccttgaggcgggcctgcccctccaggtccgaggctaccccgaggagaagaaagaggaggagggcagcgcaaaccgcagaccagaggaccaggagctggagagcctgtcggccattgaagcagagctggagaaagtggcccaccagctgcaggcactacggcggggctgagacaccggctggcagggctggccccagggcaccctgtggccctggctctgctgtccccttggcaggtcctggccagatggcccggatgctgcttccggtagggaggcagcctccagcctgcccaagcccaggccaccctatcgccccctacgcgccttgtctcctactcctgactcctacctgccctggaacatcctttgcagggcagccccacaactttaaacattgacgattccttctctgaacacaggcagctttctagaagtttcccttcctccatcctatccactgggcacaactgcaataacttctgaccttttggtgaaagctgagaactcctgactgtaacatattctgtatgaactttatctaaagaaaaataaatctgttctgggctctttcctctgaaaaaaaaaaaaaaaaaaaaaaaaaaaaaaaaaaaaaa

SEQ ID NO: 15 - protein, 677 aa, Unknown
  CHGB amino acid
MQPTLLLSLLGAVGLAAVNSMPVDNRNHNEGMVTRCIIEVLSNALSKSSAPPITPECRQVLKTSRKDVKDKETTENENTKFEVRLLRDPADASEAHESSSRGEAGAPGEEDIQGPTKADTEKWAEGGGHSRERADEPQWSLYPSDSQVSEEVKTRHSEKSQREDEEEEEGENYQKGERGEDSSEEKHLEEPGETQNAFLNERKQASAIKKEELVARSETHAAGHSQEKTHSREKSSQESGEETGSQENHPQESKGQPRSQEESEEGEEDATSEVDKRRTRPRHHHGRSRPDRSSQGGSLPSEEKGHPQEESEESNVSMASLGEKRDHHSTHYRASEEEPEYGEEIKGYPGVQAPEDLEWERYRGRGSEEYRAPRPQSEESWDEEDKRNYPSLELDKMAHGYGEESEEERGLEPGKGRHHRGRGGEPRAYFMSDTREEKRFLGEGHHRVQENQMDKARRHPQGAWKELDRNYLNYGEEGAPGKWQQQGDLQDTKENREEARFQDKQYSSHHTAEKRKRLGELFNPYYDPLQWKSSHFERRDNMNDNFLEGEEENELTLNEKNFFPEYNYDWWEKKPFSEDVNWGYEKRNLARVPKLDLKRQYDRVAQLDQLLHYRKKSAEFPDFYDSEEPVSTHQEAENEKDRADQTVLTEDEKKELENLAAMDLELQKIAEKFSQRG

SEQ ID NO: 16 - DNA, 2666 bp, Unknown
  Sequence coding CHGB polypeptide
agaccctgcacaccaggcgactggactggcgtaaatttccgcccatttgcgtcgccgcctgggggtgctgcttcgtgacgtcagggctgagctagggggcgcccgctcctggctgcccgctcctcccggccctgcggcccccgcctgccctttaaaagagcggggcctgcgccggccgcgccacaccgcggggaccaggaggcacgctggttttccggggccgctccatcgcgccttcctcctgcgcctcgcttctccggtccagccgccatcttcctttccgcacaggggccgccgagcggggccatgcagccaacgctgcttctcagcctcctgggagccgtggggctggcggctgtcaattccatgccagtggataacaggaaccacaatgaaggaatggtgactcgctgcatcattgaggtcctctcaaatgccttgtcgaagtccagcgctccacccatcacccctgagtgccgccaagtcctgaagacgagtagaaaagacgtcaaagacaaagagacaactgaaaatgaaaacacaaagtttgaagtaagattgttaagagacccagctgatgcctcggaagcccacgagtcctccagcaggggagaggcaggagccccaggggaggaggacatccaaggcccaacaaaggcagacacagagaaatgggcagagggaggcgggcacagccgagagcgagcggatgagccccagtggagcctctatccctccgacagccaagtctctgaagaagtgaagacacgccattctgagaagagccagagagaggatgaggaggaggaggagggagagaactatcaaaaaggggagcgaggggaagatagcagtgaagagaaacaccttgaagagccaggagagacacaaaacgcttttctcaatgaaagaaagcaggcttcagctataaaaaaagaggagttagtggccagatcggaaacacatgctgccgggcattctcaggagaagacacatagccgagagaagagtagccaggagagtggagaggagacagggagccaggagaatcacccccaggagtctaaaggccaaccccgaagccaggaagaatctgaggaaggtgaggaagatgccacctctgaggtggacaaacgacgcacgaggcccagacaccaccacgggaggagcaggcccgacaggtcctctcaaggagggagtcttccctctgaggaaaagggacacccccaggaggaatctgaggagtcaaacgtcagcatggccagtttaggggaaaagagggaccaccattcaacccactacagggcttcagaggaagaacctgaatatggagaagaaataaagggttatccaggcgtccaggcccctgaggacctggagtgggagcgctataggggcagaggaagtgaagaatacagggctccaagacctcagagtgaggagagttgggatgaggaggacaagagaaactaccccagcttagagcttgataagatggcacatggatatggtgaagaaagtgaggaagagaggggccttgagccgggaaagggacgccatcacagaggcaggggaggggagccacgtgcctatttcatgtctgacaccagagaagagaaaaggttcttgggtgaaggacaccaccgtgtccaagaaaaccagatggacaaggcaaggaggcatccacaaggtgcgtggaaagagctggacagaaattatctcaactacggtgaggaaggagccccagggaagtggcagcagcagggagacctgcaggacactaaagaaaacagggaggaagctaggtttcaagataaacaatatagctcccatcacacagctgaaaagaggaagagattaggggaactgttcaacccatactacgaccctctccagtggaagagcagccattttgaaagaagagacaacatgaatgacaattttctcgagggtgaggaggaaaatgagctgaccttgaacgagaagaatttcttcccagaatacaactatgactggtgggagaaaaagcccttctctgaggatgtgaactgggggtatgagaagagaaacctcgccagggtccccaagctggacctgaaaaggcaatatgacagggtggcccaactggaccagctccttcactacaggaagaagtcagctgagtttccagacttctatgattctgaggagccggtgagcacccaccaggaggcagaaaatgaaaaggacagggctgaccagacagtcctgacagaggacgagaaaaaagaactcgaaaacttggctgcaatggatttggaactacagaagatagctgagaaattcagccaaaggggctgactgtcattggagcggtgggcactgttaagaagcagccatcacatgatctgtttttcaccacttcactgaaagacaccatttatctacccaagggcagaaagtagaacttactattcattaaatgtttgacacaattggaattgtctttaatttctgtcagaatgctattgaaaatgtgaattgcatgacttgtagcatattcttttctgcaaaatagacatattaacatgcttatgacaatgactgtgctactgtctttggaaaaatgtttgtctcagttggaaataataaaagattcacctgagaccaaaaaaaaaaaaaaaaaa

SEQ ID NO: 17 - protein, 615 aa, Unknown
  VGF polypeptide
MKALRLSASALFCLLLINGLGAAPPGRPEAQPPPLSSEHKEPVAGDAVPGPKDGSAPEVRGARNSEPQDEGELFQGVDPRALAAVLLQALDRPASPPAPSGSQQGPEEEAAEALLTETVRSQTHSLPAPESPEPAAPPRPQTPENGPEASDPSEELEALASLLQELRDFSPSSAKRQQETAAAETETRTHTLTRVNLESPGPERVWRASWGEFQARVPERAPLPPPAPSQFQARMPDSGPLPETHKFGEGVSSPKTHLGEALAPLSKAYQGVAAPFPKARRPESALLGGSEAGERLLQQGLAQVEAGRRQAEATRQAAAQEERLADLASDLLLQYLLQGGARQRGLGGRGLQEAAEERESAREEEEAEQERRGGEERVGEEDEEAAEAEAEAEEAERARQNALLFAEEEDGEAGAEDKRSQEETPGHRRKEAEGTEEGGEEEDDEEMDPQTIDSLIELSTKLHLPADDVVSIIEEVEEKRKRKKNAPPEPVPPPRAAPAPTHVRSPQPPPPAPAPARDELPDWNEVLPPWDREEDEVYPPGPYHPFPNYIRPRTLQPPSALRRRHYHHALPPSRHYPGREAQARRAQEEAEAEERRLQEQEELENYIEHVLLRRP

SEQ ID NO: 18 - DNA, 2586 bp, Unknown
  Sequence coding VGF polypeptide
ccagcgtgctgaagccggagcgagctagccgcccggagccgcgccgacccagctgagcccagcccacgggacgccagacctcgaccgtcgctcctaccccggccaccgctcggagccgaggcggacgcgtcccgatcttcccctgtccccaccctgccccgaccctcctctccacctctcgcgtcgtgacaccagctgtctccggcagcctcttggtcatgaaagccctcagattgtcggcttccgccctcttctgccttctgctgatcaacgggttaggggcagcaccccctggtcgccctgaggcgcagcctcctcctctcagctctgagcataaagagccggtagccggggacgcagtgcccgggccaaaggatggcagcgccccagaggtccgaggcgctcggaattccgagccgcaggacgagggagagcttttccagggcgtggatccccgggcgctggccgcggtgctgctgcaggcactcgaccgtcccgcctcacccccggcaccaagcggctcccagcaggggccggaggaagaagcagctgaagctctgctgaccgagaccgtgcgcagccagacccacagcctcccggcgccggagagcccggagcccgcggctccgcctcgccctcagactccggagaatgggcccgaggcgagcgatccctccgaggagctcgaggcgctagcgtccctgctccaggaactgcgagatttcagtccaagtagcgccaagcgccagcaggagacggcggcagcagagacggaaacccgcacgcacacgctgacccgagtgaatctggagagcccggggccagagcgcgtatggcgcgcttcctggggagagttccaggcgcgtgtcccggagcgcgcgcccctgccgcccccggccccctctcaattccaggcgcgtatgcccgacagcgggccccttcccgaaacccacaagttcggggaaggagtgtcctcccccaaaacacacctaggcgaggcattggcacccctgtccaaggcgtaccaaggcgtggccgccccgttccccaaggcgcgccggccggagagcgcactcctgggcggctccgaggcgggcgagcgccttctccagcaagggctggcgcaggtggaggccgggcggcggcaggcggaggccacgcggcaggccgcggcgcaggaagagcggctggccgacctcgcctcggacctgctgctccagtatttgctgcagggcggggcccggcagcgcggcctcgggggtcgggggctgcaggaggcggcggaggagcgagagagtgcaagggaggaggaggaggcggagcaggagagacgcggcggggaggagagggtgggggaagaggatgaggaggcggccgaggcggaggcagaggcggaggaggcggagagggcgcggcagaacgcgctcctgttcgcggaggaggaggacggggaagccggcgccgaggacaagcgctcccaggaggagacgccgggccaccggcggaaggaggccgaggggacagaggagggcggggaggaggaggacgacgaggagatggatccgcagacgatcgacagcctcattgagctgtccaccaaactccacctgccagcggacgacgtggtcagcatcatcgaggaggtggaggagaagcggaagcggaagaagaacgcccctcccgagcccgtgccgcccccccgtgccgcccccgcccccacccacgtccgctccccgcagcccccgccccccgcccccgctcccgcacgagacgagctgccggactggaacgaggtgctcccgccctgggatcgggaggaggacgaggtgtacccgccagggccgtaccaccctttccccaactacatccggccgcggacactgcagccgccctcggccttgcgccgccgccactaccaccacgccttgccgccttcgcgccactatcccggccgggaggcccaggcgcggcgcgcgcaggaggaggcggaggcggaggagcgccggctgcaggagcaggaggagctggagaattacatcgagcacgtgctgctccggcgcccgtgactgcccttcccggtcccgcccccgcgcgcccccgccgcgcgcgcgcgccggcgcccccctccgtgttgcccgctccccctcggtgtttgcatgcgccccggccctgccccttggccctgcccctgtccccgggctgcgtcgggacctgccagacccccctcccgggtcctgagcccgaactcccagagctcacccgcgggtgaccgggggccagcccaggagggcgggtggtttgtgcgagttcccttgccacgcggggccccggccccatcaagtccctctggggacgtccccgtcggaaaccggaaaaagcagttccagttaattgtgtgaagtgtgtctgtctccagcccttcgggcctcccacgagcccctccagcctctccaagtcgctgtgaattgaccccttctttcctttctctgttgtaaatacccctcacggaggaaatagttttgctaagaaataaaagtgactattttattaggaaaaaaaaaaaaaaaaaaaaaaaaaaaaaaaaaa

SEQ ID NO: 19 - DNA, 24 bp, Artificial Sequence
  NTM Forward primer
ccctacgtctcaattcatcataag

SEQ ID NO: 20 - DNA, 24 bp, Artificial Sequence
  NTM Reverse primer
tacgatactgttgattactaaagc

SEQ ID NO: 21 - DNA, 18 bp, Artificial Sequence
  PAM Forward primer
ccctatcccagccctatc

SEQ ID NO: 22 - DNA, 20 bp, Artificial Sequence
  PAM Reverse primer
gtgatgcccaggctgagatc

SEQ ID NO: 23 - DNA, 18 bp, Artificial Sequence
  PTPRN2 Forward primer
ctggtctgcccctctcct

SEQ ID NO: 24 - DNA, 22 bp, Artificial Sequence
  PTPRN2 Rerverse primer
ttcaggagattttcatccttcc

SEQ ID NO: 25 - DNA, 21 bp, Artificial Sequence
  LY6H F
ctgctggccgtcctgctgtgc

SEQ ID NO: 26 - DNA, 23 bp, Artificial Sequence
  LY6H R
ccaacttactgctgctgggatcc

SEQ ID NO: 27 - DNA, 20 bp, Artificial Sequence
  OPCML Forward primer
cctaggtcctctgagcaacg

SEQ ID NO: 28 - DNA, 20 bp, Artificial Sequence
  OPCML Reverse primer
ggtcaaggtagcaggagcag

SEQ ID NO: 29 - DNA, 22 bp, Artificial Sequence
  YWHAZ Forward primer
gcctgcatgaagtctgtaactg

SEQ ID NO: 30 - DNA, 21 bp, Artificial Sequence
  YWHAZ Reverse primer
tgacctacgggctcctacaac

SEQ ID NO: 31 - DNA, 18 bp, Artificial Sequence
  CHGA Forward primer
cctgtgaacagccctatg

SEQ ID NO: 32 - DNA, 18 bp, Artificial Sequence
  CHGA Reverse primer
ggaaagtgtgtcggagat

SEQ ID NO: 33 - DNA, 21 bp, Artificial Sequence
  CHGB Forward primer
caactggaccagctccttcac

SEQ ID NO: 34 - DNA, 24 bp, Artificial Sequence
  CHGB Reverse primer
gcacagtcattgtcataagcatgt

SEQ ID NO: 35 - DNA, 18 bp, Artificial Sequence
  VGF Forward primer
cctcttggtcatgaaagc

SEQ ID NO: 36 - DNA, 18 bp, Artificial Sequence
  VGF Reverse primer
ggctctttatgctcagag

(19) United States
(12) Patent Application Publication
(10) Pub. No.: US 2022/0003787 A1
(43) Pub. Date: Jan. 6, 2022

(54) BIOMARKER PROTEINS FOR DIAGNOSING ALZHEIMER'S DEMENTIA AND USE THEREOF

(71) Applicant: AJOU UNIVERSITY INDUSTRY-ACADEMIC COOPERATION FOUNDATION, Suwon-si, Gyeonggi-do (KR)

(72) Inventor: Sun Ah PARK, Seoul (KR)

(73) Assignee: AJOU UNIVERSITY INDUSTRY-ACADEMIC COOPERATION FOUNDATION, Suwon-si, Gyeonggi-do (KR)

(21) Appl. No.: 17/290,016

(22) PCT Filed: Sep. 11, 2019
(86) PCT No.: PCT/KR2019/011862

§ 371 Date: Apr. 29, 2021

(30) Foreign Application Priority Data
Oct. 30, 2018 (KR) 1020180131309
Mar. 13, 2019 (KR) 1020190028903

(51) Int. Cl. G01N 33/68; C12Q 1/6883

(52) U.S. Cl. CPC G01N 33/6896; C12Q 1/6883

(57) ABSTRACT

Provided are a composition for diagnosing Alzheimer's dementia, the composition including an agent for measuring levels of mRNAs of one or more genes selected from the group consisting of neurotrimin (NTM), peptidylglycine-α-amidating monooxygenase (PAM), receptor-type tyrosine-protein phosphatase N2 (PTPRN2), opioid-binding protein/cell adhesion molecule (OPCML), 14-3-3 protein ζ/δ (YWHAZ), chromogranin-A (CHGA), and secretogranin-1 (CHGB), or levels of proteins expressed therefrom; a kit for diagnosing Alzheimer's dementia, the kit including the composition; and a method of providing information for the diagnosis of Alzheimer's dementia, the method including the steps of measuring the levels of mRNAs of the genes or the levels of proteins expressed therefrom, and comparing the measured levels with those measured in a sample isolated from a normal individual.

The composition for diagnosing Alzheimer's dementia of the present disclosure may be used to diagnose Alzheimer's dementia with high sensitivity and specificity by measuring expression levels of mRNAs of the genes or expression levels of proteins expressed therefrom, and then comparing the levels with those of a normal control group.

### BACKGROUND OF THE INVENTION

### 1. Field of the Invention

The present disclosure relates to a composition for diagnosing Alzheimer's dementia, the composition including an agent for measuring levels of mRNAs of one or more genes selected from the group consisting of neurotrimin (NTM), peptidylglycine-α-amidating monooxygenase (PAM), receptor-type tyrosine-protein phosphatase N2 (PTPRN2), opioid-binding protein/cell adhesion molecule (OPCML), 14-3-3 protein ζ/δ (YWHAZ), chromogranin-A (CHGA), and secretogranin-1 (CHGB), or levels of proteins expressed therefrom; a kit for diagnosing Alzheimer's dementia, the kit including the composition; and a method of providing information for the diagnosis of Alzheimer's dementia, the method including the steps of measuring the levels of mRNAs of the genes or the levels of proteins expressed therefrom, and then comparing the measured levels with those measured in a sample isolated from a normal individual.

### 2. Description of the Related Art

The increasing number of dementia patients due to the super-aging phenomenon worldwide is currently emerging as a serious social problem. According to the Korean National Assembly Budget Office in 2014, the prevalence of dementia among individuals aged 65 and older is expected to increase to 840,000 in 2020 and 2.17 million in 2050, and accordingly, the scale of social costs due to dementia is also expected to rapidly increase from 11.7 trillion won in 2013 to 23.1 trillion won in 2030, 34.2 trillion won in 2040, and 43.2 trillion won in 2050.

Currently, diagnostic methods used to diagnose Alzheimer's dementia include a genetic test, neuropsychological and cognitive tests, a cerebrospinal fluid test, and a brain imaging test (MRI, PET). The genetic test is a test that detects the risk of Alzheimer's dementia through a test for an Alzheimer's dementia-specific gene called ApoE4. However, ApoE4 gene may increase the risk of dementia, but it is not a crucial factor. Thus, it is difficult to diagnose Alzheimer's dementia only with this test. Further, the neuropsychological and cognitive tests are methods of measuring the degree of cognitive impairment of a patient through questionnaires, and include Mini-Mental State Examination (MMSE), Montreal Cognitive Assessment (MoCA), SNSB, etc. This is a questionnaire-type diagnosis method which has an advantage of low cost and no physical pain during diagnosis. However, there are problems such as changes in results due to differences in learning, age, and educational background due to repeated tests, accuracy due to subjective intervention, etc. Critically, there is a problem in that it is impossible to determine whether the cognitive impairment is caused by beta-amyloid, which is a major factor of Alzheimer's dementia, or whether it is caused by other types of diseases. Further, the cerebrospinal fluid test is a method of quantitatively diagnosing beta-amyloid or tau protein, which is known as an Alzheimer's disease-causing factor, by extracting cerebrospinal fluid. Although the accuracy of the diagnosis is high, a lumbar puncture to obtain cerebrospinal fluid may cause great pain to patients, which may cause patients' refusal. In addition, it is difficult to perform the lumbar puncture in general hospitals that do not have high expertise in the procedure, and there are cost limitations. The brain imaging test (MRI, PET) is a method of analyzing the degree of brain damage, and beta-amyloid and tau protein, which are Alzheimer's disease-causing factors, in the brain through MRI or PET imaging. Although this method has high accuracy for diagnosis, it requires expensive equipment and expertise in imaging when diagnosing Alzheimer's dementia. Thus, there is a problem in that distribution is limited and high diagnostic costs are required. As described, there are various techniques for diagnosing Alzheimer's dementia, but each technique has various limitations such as accuracy problems, cost problems, time constraints, physical pain, etc.

The present inventors found that expression levels of seven kinds of genes or proteins (neurotrimin (NTM), peptidylglycine-α-amidating monooxygenase (PAM), receptor-type tyrosine-protein phosphatase N2 (PTPRN2), opioid-binding protein/cell adhesion molecule (OPCML), 14-3-3 protein ζ/δ (YWHAZ), chromogranin-A (CHGA), and secretogranin-1 (CHGB)) derived from cerebrospinal fluid show a significant difference between a patient with Alzheimer's dementia and a normal control group, and information related to diagnosis or prognosis of Alzheimer's dementia may be more accurately obtained by simultaneously comparing the levels of the seven kinds of the genes or proteins, more specifically three kinds of genes or proteins (YWHAZ, CHGA, and CHGB), thereby completing the present disclosure.

### SUMMARY OF THE INVENTION

An object of the present disclosure is to provide a composition for diagnosing Alzheimer's dementia, the composition including an agent for measuring levels of mRNAs of one or more genes selected from the group consisting of neurotrimin (NTM), peptidylglycine-α-amidating monooxygenase (PAM), receptor-type tyrosine-protein phosphatase N2 (PTPRN2), opioid-binding protein/cell adhesion molecule (OPCML), 14-3-3 protein ζ/δ (YWHAZ), chromogranin-A (CHGA), and secretogranin-1 (CHGB), or levels of proteins expressed therefrom.

Another object of the present disclosure is to provide a kit for diagnosing Alzheimer's dementia, the kit including the composition.

Still another object of the present disclosure is to provide a method of providing information for the diagnosis of Alzheimer's dementia, the method including the steps of (a) measuring levels of mRNAs of one or more genes selected from the group consisting of NTM, PAM, PTPRN2, OPCML, YWHAZ, CHGA, and CHGB, or levels of proteins expressed therefrom in a sample isolated from an individual suspected of having Alzheimer's dementia; and (b) comparing the measured levels with those measured in a sample isolated from a normal individual.

### BRIEF DESCRIPTION OF THE DRAWINGS

FIG. 1 shows a schematic illustration of a high-efficiency proteomic analysis process capable of analyzing nine kinds of proteins (NTM, PAM, PTPRN2, LY6H, OPCML, YWHAZ, CHGA, CHGB, and VGF) in cerebrospinal fluids;

FIG. 2 shows results of analyzing Alzheimer's dementia diagnostic utility of individual proteins and a combination of three kinds of proteins (YWHAZ, CHGA, and CHGB) through regression equation values (Equation, red), wherein ROC curve analysis showed that diagnostic utility may be increased, through the regression equation values obtained by a combination of three kinds of proteins (“combine” in the graph);

FIG. 3 is a graph showing a correlation between the protein expression levels and the mini-mental state examination (MMSE) values capable of evaluating cognitive impairment caused by Alzheimer's disease, wherein a combination of three proteins (YWHAZ, CHGA, and CHGB) is indicated by “combined” in the graph; and

FIG. 4 is a graph showing a correlation between the protein expression levels and the clinical dementia rating-sum-of-box (CDR-SOB) values capable of clinically evaluating the severity of dementia, wherein a combination of three proteins (YWHAZ, CHGA, and CHGB) is indicated by “combined” in the graph.

### DETAILED DESCRIPTION OF THE PREFERRED EMBODIMENTS

The present disclosure will be described in detail as follows. Meanwhile, each description and embodiment disclosed in this disclosure may also be applied to other descriptions and embodiments. That is, all combinations of various elements disclosed in this disclosure fall within the scope of the present disclosure. Further, the scope of the present disclosure is not limited by the specific description described below.

To achieve the above objects, one aspect of the present disclosure provides a composition for diagnosing Alzheimer's dementia, the composition including an agent for measuring levels of mRNA(s) of one or more genes selected from the group consisting of neurotrimin (NTM), peptidylglycine-α-amidating monooxygenase (PAM), receptor-type tyrosine-protein phosphatase N2 (PTPRN2), opioid-binding protein/cell adhesion molecule (OPCML), 14-3-3 protein ζ/δ (YWHAZ), chromogranin-A (CHGA), and secretogranin-1 (CHGB), or levels of proteins expressed therefrom.

The composition may include one kind of gene, or a combination of two, three, four, five, six, or seven kinds of genes among the seven kinds of genes. Specifically, the composition may include a combination of YWHAZ, CHGA, and CHGB genes, but is not limited thereto.

In addition, the composition may further include any one or more genes selected from the group consisting of NTM, PAM, lymphocyte antigen 6 family member H (LY6H), PTPRN2, OPCML, and VGF (neurosecretory protein VGF), in addition to a combination of the three kinds of genes, but is not limited thereto. Specifically, the composition may further include one or more genes of lymphocyte antigen 6 family member H (LY6H), neurosecretory protein VGF, and OPCML, but is not limited thereto.

The composition of the present disclosure may be used to obtain information useful for diagnosis or prognosis of Alzheimer's dementia by examining a significant difference in the gene expression between a patient with Alzheimer's dementia and a normal individual. This has not yet been known, and was first revealed by the present inventors, and it has great significance in that it is possible to diagnose or prognose Alzheimer's dementia with high sensitivity and specificity.

As used herein, the term “NTM” gene refers to a gene encoding neurotrimin, and which is known to be closely related to neurite outgrowth, intercellular connection between neurons, and adhesion by a specific antibody mechanism. Specifically, the neurotrimin may be a protein including an amino acid sequence having activity of neurotrimin encoded by the NTM gene, and the amino acid sequence may consist of SEQ ID NO: 1, but is not limited thereto. Further, mRNA of the NTM gene may consist of a nucleotide sequence of SEQ ID NO: 2, but is not limited thereto.

As used herein, the term “PAM” gene refers to a gene encoding peptidylglycine-α-amidating monooxygenase, and which is known as an enzyme that enhances synaptic function, regulates neurobiological function through C-terminal amidation, and α-amidates the carboxyl terminal to impart activity to inactive proteins in vivo. Specifically, the peptidylglycine-α-amidating monooxygenase may be a protein including an amino acid sequence having activity of peptidylglycine-α-amidating monooxygenase encoded by the PAM gene, and the amino acid sequence may consist of SEQ ID NO: 3, but is not limited thereto. Further, mRNA of the PAM gene may consist of a nucleotide sequence of SEQ ID NO: 4, but is not limited thereto.

As used herein, the “PTPRN2” gene is also known as islet cell autoantigen-related protein (ICAAR) or phogrin, and is a gene encoding receptor-type tyrosine-protein phosphatase N2 enzyme. The gene is known to have an important role in the release of neurosecretory substances through the endoplasmic reticulum, regulation of protein phosphorylation, and maintenance of the neuronal skeleton. Specifically, the ICAAR may be a protein including an amino acid sequence having activity of ICAAR encoded by the PTPRN2 gene, and the amino acid sequence may consist of SEQ ID NO: 5, but is not limited thereto. Further, mRNA of the PTPRN2 gene may consist of a nucleotide sequence of SEQ ID NO: 6, but is not limited thereto.

As used herein, the term “LY6H” gene refers to cDNA containing a 420-codon open reading frame (ORF) encoding lymphocyte antigen 6 family member H (LY6H), which is a novel brain-specific protein, and having a full-length sequence of 854 nucleotides, and which is known to regulate expression and function of acetylcholine receptor, which has a vital role in cognitive function. Specifically, the LY6H protein may be a protein including an amino acid sequence having activity of LY6H encoded by the LY6H gene, and the amino acid sequence may consist of SEQ ID NO: 7, but is not limited thereto. Further, mRNA of the LY6H gene may consist of a nucleotide sequence of SEQ ID NO: 8, but is not limited thereto.

As used herein, the term “OPCML” gene refers to a gene encoding opioid-binding protein/cell adhesion molecule, which is highly conserved in species during evolution, and thus may have a fundamental role in mammalian systems. Specifically, the opioid-binding protein/cell adhesion molecule may be a protein including an amino acid sequence having activity of opioid-binding protein/cell adhesion molecule encoded by the OPCML gene, and the amino acid sequence may consist of SEQ ID NO: 9, but is not limited thereto. Further, mRNA of the OPCML gene may consist of a nucleotide sequence of SEQ ID NO: 10, but is not limited thereto.

As used herein, the term “YWHAZ(1433Z)” gene refers to a gene encoding 14-3-3 protein zeta/delta (14-3-3 proteins ζ/δ), which is a protein belonging to conserved regulatory molecules expressed in all eukaryotic cells, and 14-3-3 protein may bind to various signal proteins, including kinases, phosphatase, and transmembrane receptors, and 200 or more signal proteins are known as 14-3-3 ligands. Further, the amount of 14-3-3 protein is found to increase in the cerebrospinal fluid (CSF) of patients with Creutzfeldt-Jakob disease. Specifically, the 14-3-3 protein may be a protein including an amino acid sequence having activity of 14-3-3 protein encoded by the YWHAZ gene, and the amino acid sequence may consist of SEQ ID NO: 11, but is not limited thereto. Further, mRNA of the YWHAZ gene may consist of a nucleotide sequence of SEQ ID NO: 12, but is not limited thereto.

As used herein, the term “CHGA” gene refers to a gene encoding chromogranin A, also called parathyroid secretory protein 1, which is a protein belonging to the granin family of neuroendocrine secretory proteins. Chromogranin A is located in secretory vesicles of neurons and endocrine cells such as islet beta cell secretory granules in the pancreas. It has also been reported that chromogranin A may be used as an indicator for pancreatic cancer and prostate cancer. Specifically, the chromogranin A may be a protein including an amino acid sequence having activity of chromogranin A encoded by the CHGA gene, and the amino acid sequence may consist of SEQ ID NO: 13, but is not limited thereto. Further, mRNA of the CHGA gene may consist of a nucleotide sequence of SEQ ID NO: 14, but is not limited thereto.

As used herein, the term “CHGB” gene refers to a gene encoding chromogranin B, also called secretogranin I, which is a protein belonging to the granin family of neuroendocrine secretory proteins. It has been reported that chromogranin B may be used as a prognostic biomarker for neuroendocrine tumors. Specifically, the chromogranin B may be a protein including an amino acid sequence having activity of chromogranin B encoded by the CHGB gene, and the amino acid sequence may consist of SEQ ID NO: 15, but is not limited thereto. Further, mRNA of the CHGB gene may consist of a nucleotide sequence of SEQ ID NO: 16, but is not limited thereto.

As used herein, the term “VGF” gene refers to a gene encoding neurosecretory protein VGF, and the neurosecretory protein VGF is known to have an important role in regulating energy homeostasis, metabolism, and synaptic plasticity. Specifically, the neurosecretory protein VGF may be a protein including an amino acid sequence having activity of neurosecretory protein VGF encoded by the VGF gene, and the amino acid sequence may consist of SEQ ID NO: 17, but is not limited thereto. Further, mRNA of the VGF gene may consist of a nucleotide sequence of SEQ ID NO: 18, but is not limited thereto.

The nine kinds of proteins or mRNAs of the genes according to the present disclosure include the amino acid sequences consisting of SEQ ID NOs: 1, 3, 5, 7, 9, 11, 13, 15, and 17, and nucleotide sequences consisting of SEQ ID NOs: 2, 4, 6, 8, 10, 12, 14, 16, and 18, respectively, and also include any amino acid sequences or nucleotide sequences without limitation, as long as they have 80% or more, specifically 90% or more, more specifically 95% or more, much more specifically 98% or more, and most specifically 99% or more homology to the above sequences, and exhibit efficacy substantially identical or corresponding to those of the proteins or genes. Additionally, it is obvious that any amino acid sequence or nucleotide sequence with deletion, modification, substitution, or addition in part of the sequence may also be included within the scope of the present disclosure, as long as the amino acid sequence or nucleotide sequence has the homology described above.

As used herein, the term “homology” refers to a percentage of identity between two given polynucleotides or polypeptide moieties. Sequence homology from one moiety to another moiety may be determined by a known technique in the art. For example, homology may be determined by directly aligning the sequence information of two polynucleotide molecules or two polypeptide molecules using a computer program that is readily available and capable of aligning sequence information. The computer program may include BLAST (NCBI), CLC Main Workbench (CLC bio), MegAlign™ (DNASTAR Inc), etc., but any program may be used without limitation, as long as it is a program capable of determining homology. In addition, homology between polynucleotides may be determined by hybridizing the polynucleotides under the conditions for forming a stable double strand in the homologous regions, and then digesting the hybridized strand by a single-strand-specific nuclease to determine a size of a digested fragment, but is not limited thereto.

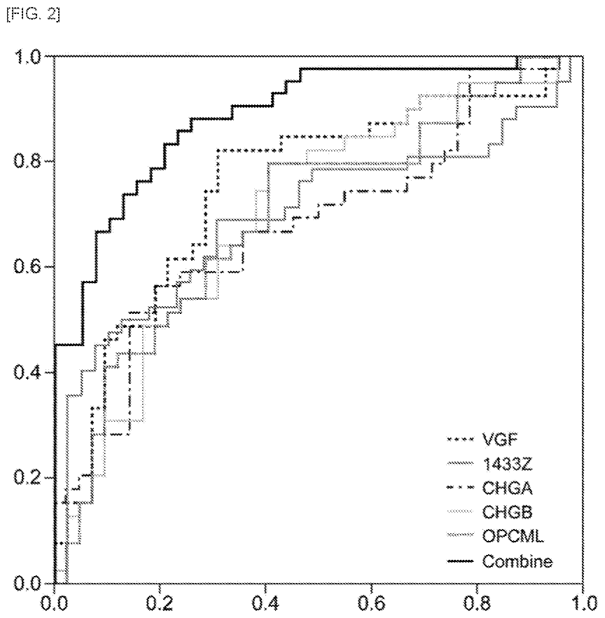

In one exemplary embodiment of the present disclosure, nine kinds of proteins (NTM, PAM, PTPRN2, LY6H, VGF, YWHAZ, CHGA, CHGB, and OPCML) which show significant differences in expression levels between a normal individual and a patient with Alzheimer's dementia were identified, and it was found that when three kinds of proteins (YWHAZ, CHGA, and CHGB) among them are measured at the same time, it is possible to diagnose Alzheimer's dementia with high sensitivity and specificity. As a result, it was confirmed that levels of mRNAs of the three kinds of proteins or levels of proteins expressed therefrom may be measured to be effectively used in diagnosing Alzheimer's dementia (FIG. 2).

As used herein, the term “agent for measuring levels of mRNAs” refers to an agent used in a method of measuring levels of mRNAs transcribed from the genes, in order to examine expression of the nine kinds of genes of the present disclosure which are included in a sample. Specifically, the agent may include a primer or a probe capable of specifically binding to a target gene used in RT-PCR, quantified real-time PCR, Competitive RT-PCR, real-time quantitative RT-PCR, RNase protection assay (RPA), Northern blotting, DNA chip analysis, etc., but is not limited thereto.

The “primer” is a short nucleic acid sequence having a short free 3′ hydroxyl group, which may form a base pair with a complementary template, and function as a starting point for the replication of a template strand. A primer may initiate DNA synthesis in the presence of reagents and four kinds of nucleoside triphosphates for polymerization (i.e., DNA polymerase or reverse transcriptase) in an appropriate buffer at an appropriate temperature.

Specifically, each of the primer sequences used in the method of measuring mRNA expression levels of the nine kinds of genes are shown in Table 1 below.

[TABLE 1]

| | |
|---|---|
| NTM (neurotrimin) | Forward: |
| UniProtID: Q9P121 | CCCTACGTCTCAATTCAT |
| Gene ID: 50863 | CATAAG |
| | (SEQ ID NO: 19) |
| | Backward: |
| | TACGATACTGTTGATTAC |
| | TAAAGC |
| | (SEQ ID NO: 20) |
| PAM (Peptidylglycine-α | Forward: |
| amidating monooxygenase) | CCCTATCCCAGCCCTATC |
| UniProt ID: P19021 | (SEQ ID NO: 21) |
| Gene ID: 5066 | Backward: |
| | GTGATGCCCAGGCTGAGATC |
| | (SEQ ID NO: 22) |
| PTPRN2 (receptor-type | Forward: |
| tyrosine-protein | CTGGTCTGCCCCTCTCCT |
| phosphatase N2) | (SEQ ID NO: 23) |
| UniProt ID: Q92932 | Backward: |
| Gene ID: 5799 | TTCAGGAGATTTTCATCCTTCC |
| | (SEQ ID NO: 24) |
| LY6H (lymphocyte | Forward: |
| antigen 6H) | CTGGTGGCCGTCCTGCTGTGC |
| UniProt ID: O94772 | (SEQ ID NO: 25) |
| Gene ID: 4062 | Backward: |
| | CCAACTTACTGCTGCTGG |
| | GATCC |
| | (SEQ ID NO: 26) |
| OPCML | F: |
| | CCTAGGTCCTCTGAGCAACG |
| | (SEQ ID NO: 27) |
| | R: |
| | GGTCAAGGTAGCAGGAGCAG |
| | (SEQ ID NO: 28) |
| YWHAZ | F: |
| | GCCTGCATGAAGTCTGTA |
| | ACTG |
| | (SEQ ID NO: 29) |
| | R: |
| | TGACCTACGGGCTCCTACAAC |
| | (SEQ ID NO: 30) |
| CHGA | F: |
| | CCTGTGAACAGCCCTAG |
| | (SEQ ID NO: 31) |
| | R: |
| | GGAAAGTGTGTCGGAGAT |
| | (SEQ ID NO: 32) |
| CHGB | F: |
| | CAACTGGACCAGCTCCTTCAC |
| | (SEQ ID NO: 33) |
| | R: |
| | GCACAGTCATTGTCATAA |
| | GCATGT |
| | (SEQ ID NO: 34) |
| VGF | F: |
| | CCTCTTGGTCATGAAAGC |
| | (SEQ ID NO: 35) |
| | R: |
| | GGCTCTTTATGCTCAGAG |
| | (SEQ ID NO: 36) |

Meanwhile, the “probe” may be a probe capable of complementarily binding to the gene, and a nucleotide sequence of the probe is not limited, as long as it is able to complementarily bind to each gene.

As used herein, the term “agent for measuring levels of proteins” refers to an agent used in a method of measuring expression levels of proteins encoded by the nine kinds of genes of the present disclosure which are included in a sample. Specifically, the agent for measuring levels of proteins may include antibodies or aptamers specific to the proteins. Specifically, the agent may include antibodies used in Western blotting, enzyme-linked immunosorbent assay (ELISA), radioimmunoassay (RIA), radioimmunodiffusion, Ouchterlony immunodiffusion, rocket immunoelectrophoresis, immunohistochemical staining, immunoprecipitation assay, complement fixation assay, immunofluorescence, immunochromatography, fluorescence-activated cell sorter analysis (FACS), protein chip technology assay, etc., but is not limited thereto.

The “antibody” refers to a proteinaceous molecule capable of specifically binding to an antigenic region of a protein or peptide molecule. Such an antibody may be prepared by cloning each gene into an expression vector according to a common method to obtain a protein encoded by the marker gene, and producing the same from the obtained protein by a common method. The structure of the antibody is not particularly limited, and polyclonal antibodies, monoclonal antibodies, or a part thereof having an antigen-binding property may be included in the antibody of the present disclosure. In addition to all immunoglobulin antibodies, the antibody may also include specialized antibodies such as humanized antibodies, etc. Furthermore, the antibody also includes a functional fragment of an antibody molecule as well as an intact form having two full-length light chains and two full-length heavy chains. The functional fragment of an antibody molecule means a fragment having at least an antigen-binding function, and may include Fab, F(ab′), F(ab′)₂, Fv, etc.

The “aptamer” means a single-stranded oligonucleotide, and refers to a nucleic acid molecule having binding activity for a predetermined target molecule. The aptamer may have various three-dimensional structures depending on a nucleotide sequence thereof, and may have high affinity for a specific substance such as an antigen-antibody reaction. The aptamer may inhibit activity of a predetermined target molecule by binding to the predetermined target molecule.

The aptamer of the present disclosure may be RNA, DNA, a modified nucleic acid, or a mixture thereof, and its shape may be in a linear or circular form, but is not limited thereto. The aptamer of the present disclosure may be used for the proteins encoded by the nine kinds of genes. The aptamer having binding activity for the protein may be easily prepared by those skilled in the art according to a known method with reference to each nucleotide sequence.

As used herein, the term “Alzheimer's dementia” refers to a dementia symptom caused by Alzheimer's disease. Alzheimer's disease refers to the most common degenerative brain disease that causes dementia, first described by German doctor Alzheimer, and it is known that as Alzheimer's disease progresses, overall cognitive function including memory is gradually weakened.

As used herein, the term “diagnosis” refers to determining susceptibility of an individual to a specific disease or disorder, determining whether an individual has a specific disease or disorder, or monitoring the condition of an individual to provide information on therapeutic efficacy. With respect to the objects of the present disclosure, diagnosis is to determine the onset of Alzheimer's dementia or the stage of Alzheimer's dementia.

Another aspect of the present disclosure provides a kit for diagnosing Alzheimer's dementia, the kit including the composition.

The terms “Alzheimer's dementia”, “diagnosis”, etc. are the same as described above.

The kit of the present disclosure may be used for measuring expression levels of mRNAs of NTM, PAM, PTPRN2, LY6H, OPCML, YWHAZ, CHGA, CHGB, and VGF genes and levels of proteins expressed therefrom in order to diagnose Alzheimer's dementia.

The kit of the present disclosure may be a reverse transcription polymerase chain reaction (RT-PCR) kit, a DNA chip kit, an enzyme-linked immunosorbent assay (ELISA) kit, a protein chip kit, a rapid kit, or a multiple reaction monitoring (MRM) kit, but is not limited thereto.

Specifically, the kit for measuring mRNA expression levels of the genes of the present disclosure may be a kit including essential elements required for performing RT-PCR. The RT-PCR kit may include each primer pair specific to the gene, a test tube or an appropriate container, a reaction buffer (with various pHs and magnesium concentrations), deoxynucleotides (dNTPs), an enzyme such as Taq-polymerase and reverse transcriptase, a DNase, an RNase inhibitor, DEPC-water, sterile water, etc. The kit may further include a primer pair specific to a gene used as a quantitative control.

Further, the kit of the present disclosure may include essential elements required for performing DNA chip analysis. The kit for the DNA chip analysis may include a substrate on which a cDNA corresponding to a gene or a fragment thereof is attached as a probe, a reagent for preparing a fluorescent-labeled probe, a formulation, an enzyme, etc. The substrate may include a cDNA corresponding to a quantitative control gene or a fragment thereof.

Furthermore, the kit of the present disclosure may be a kit for protein chip analysis for measuring the levels of the proteins encoded by the genes. The kit may include, but is not particularly limited to, a substrate for immunological detection of an antibody, an appropriate buffer solution, a secondary antibody labeled with a chromogenic enzyme or fluorescent substance, a chromogenic substrate, etc. The substrate may be, but is not particularly limited to, a nitrocellulose membrane, a 96-well plate synthesized with a polyvinyl resin, a 96-well plate synthesized from a polystyrene resin, a slide glass made of glass, etc. The chromogenic enzyme may be, but is not particularly limited to, a peroxidase or an alkaline phosphatase. The fluorescent substance may be, but is not particularly limited to, FITC, RITC, etc. The chromogenic substrate may be, but is not particularly limited to, 2,2′-azino-bis(3-ethylbenzothiazoline-6-sulfonic acid) (ABTS), o-phenylenediamine (OPD), tetramethylbenzidine (TMB), etc.

Still another aspect of the present disclosure provides a method of providing information for the diagnosis of Alzheimer's dementia, the method including the steps of (a) measuring levels of mRNAs of one or more genes selected from the group consisting of NTM, PAM, PTPRN2, OPCML, YWHAZ, CHGA, and CHGB, or levels of proteins expressed therefrom in a sample isolated from an individual suspected of having Alzheimer's dementia; and (b) comparing the measured levels with those measured in a sample isolated from a normal individual.

The terms “Alzheimer's dementia”, “diagnosis”, etc. are the same as described above.

As used herein, the term “individual” refers to a target for diagnosing Alzheimer's dementia or for predicting prognosis of Alzheimer's dementia. In this regard, the individual may be any animal without limitation, as long as it is an animal that may develop Alzheimer's dementia, such as humans, dogs, horses, cows, mice, goats, rabbits, chickens, ducks, geese, etc.

As used herein, the term “sample” may include, but is not limited to, a tissue, a cell, whole blood, serum, plasma, saliva, sputum, cerebrospinal fluid, urine, etc., which is isolated from an individual suspected of having Alzheimer's dementia, and specifically, the sample may refer to cerebrospinal fluid.

As used herein, the term “normal individual” refers to an individual not diagnosed to have Alzheimer's dementia, and it is possible to accurately predict occurrence of Alzheimer's dementia or to predict prognosis of Alzheimer's dementia in an individual suspected of having Alzheimer's dementia by comparing expression levels of mRNAs of the genes or expression levels of the proteins expressed therefrom between a sample isolated from a normal individual and a sample isolated from an individual for whom Alzheimer's dementia is intended to be diagnosed or prognosed.

As used herein, the term “measuring expression levels of mRNAs” is a process of examining the presence or expression of mRNAs of marker genes in a biological sample in order to diagnose or prognose Alzheimer's dementia, and is determined by measuring the quantities of mRNAs. Analysis methods therefor may include reverse transcriptase polymerase chain reaction (RT-PCR), competitive reverse transcriptase polymerase chain reaction (competitive RT-PCR), real-time quantitative RT-PCR, an RNase protection method, Northern blotting, a DNA chip technology assay, etc.

As used herein, the term “measuring expression levels of proteins” is a process of examining the presence or expression of proteins encoded by the nine kinds of genes in a biological sample in order to diagnose or prognose Alzheimer's dementia, and the amounts of the proteins may be determined by using antibodies specifically binding to the proteins encoded by the genes. Analysis methods therefor may include Western blotting, enzyme-linked immunosorbent assay (ELISA), radioimmunoassay (RIA), radioimmunodiffusion, Ouchterlony immunodiffusion, rocket immunoelectrophoresis, immunohistochemical staining, immunoprecipitation assay, complement fixation assay, immunofluorescence, immunochromatography, fluorescence-activated cell sorter analysis (FACS), protein chip technology assay, etc., but is not limited thereto.

According to the method of providing information for the diagnosis of Alzheimer's dementia of the present disclosure, the expression levels of mRNAs of the genes or proteins expressed therefrom are measured in a sample isolated from an individual suspected of having Alzheimer's dementia, and when the levels are significantly high or low, as compared with those measured in a sample isolated from a normal individual, it may be determined that the risk of Alzheimer's dementia is high or the prognosis thereof is poor. Specifically, the levels of mRNAs of the genes or proteins expressed therefrom which are measured in a sample isolated from an individual suspected of having Alzheimer's dementia are compared with those measured in a sample isolated from a normal individual, and i) when the level of mRNA or protein of YWHAZ gene measured in the sample isolated from the individual suspected of having Alzheimer's dementia is high, or ii) when the level of mRNA or protein of NTM, PAM, PTPRN2, LY6H, VGF, CHGA, CHGB, or OPCML gene measured in the sample isolated from the individual suspected of having Alzheimer's dementia is low, it may be determined that the risk of Alzheimer's dementia is high, but this is not limited thereto.

Hereinafter, the construction and effects of the present disclosure will be described in more detail with reference to exemplary embodiments. However, these exemplary embodiments are for illustrative purposes only, and the scope of the present disclosure is not intended to be limited by these exemplary embodiments.

### <Example 1> Preparation of Experimental Materials and Analysis Method

### Example 1-1: Target Samples and Characteristics Thereof

To perform accurate proteomic analysis of cerebrospinal fluids obtained from Alzheimer's dementia patients (AD) and normal persons (Control), they were collected according to each condition (Park S A et al., J Clin Neurol 2015). These samples were stored in a cryogenic freezer at −80° C. until proteomic analysis. Thereafter, the samples were put in a container containing dry ice and transferred to a professional company for protein analysis, and were then used for analysis after thawing. The clinical characteristics of the subject donor are shown in Table 2 below.

TABLE 2

| | | Normal control | AD | |
|---|---|---|---|---|
| | | (n = 39) | (n = 42) | p-Value |
| Sex (male:female) | | 10:29 | 14:28 | 0.476 |
| Age at sampling | | 58.9 ± 6.3 | 60.3 ± 5.7 | 0.309 |
| Education (year) | | 10.2 ± 3.2 | 10.2 ± 4.0 | 0.962 |
| Duration of illness (year) | | — | 2.0 ± 1.2 | — |
| MMSE | | 28.3 ± 1.6 | 18.9 ± 6.4 | <0.001 |
| CDR | | 0 ± 0 | 1.1 ± 0.8 | <0.001 |
| CDR-SOB | | 0 ± 0.1 | 5.5 ± 5.3 | <0.001 |
| APOE ε4 carriers | | 12.8% | 45.2% | 0.001 |
| APOE ε4 | One allele | 5 | 13 | — |
| carriers | Two alleles | 0 | 6 | — |

| | | | |
|---|---|---|---|
| CSF Aβ42 (pg/mL) | 704.2 ± 141.4 | 348.4 ± 88.5 | <0.001 |
| CSF tTau (pg/mL) | 207.7 ± 55.3 | 637.8 ± 301.8 | <0.001 |
| CSF pTau181 (pg/mL) | 42.2 ± 12.6 | 78.3 ± 28.1 | <0.001 |

Values are shown in mean±standard deviation. p-Values are determined through either independent t-test or Chi-squared tests depending on the character of the variables.

AD, Alzheimer's dementia; APOE, apolipoprotein E; CDR-SOB, clinical dementia rating scale sum-of-boxes; CSF, cerebrospinal fluid; MMSE, mini-mental state examination.

Diagnosis of Alzheimer's dementia was based on an accurate diagnostic method, in which concentrations of beta-amyloid protein 1-42, total tau protein, and phosphorylated tau protein in cerebrospinal fluids, as well as clinical symptoms, were measured, and then when Alzheimer's dementia was diagnosed, they were classified as Alzheimer's dementia patients. In other words, the above-described method used to diagnose Alzheimer's dementia involves conditions that satisfies all AT(N) biomarkers for diagnosis of Alzheimer's disease according to the NIA-AA research framework in 2018 (Jack Jr C R et al., Alzheimer's & Dementia 2018).

### Example 1-2: Proteomic Analysis Method

Cerebrospinal fluids were collected from normal person and Alzheimer's dementia patient groups, and subjected to unbiased label-free proteomic analysis. Among proteomic analysis methods, Sequential Window Acquisition of all theoretical fragment-ion spectra (SWATH)-mass spectroscopy (MS) was used, which shows high accuracy and sensitivity in quantification and enables ‘non-target proteomic analysis’. Advantages of SWATH-MS proteomic analysis are described in the prior art (Gillet L C et al., Mol Cell Proteomics 2012).

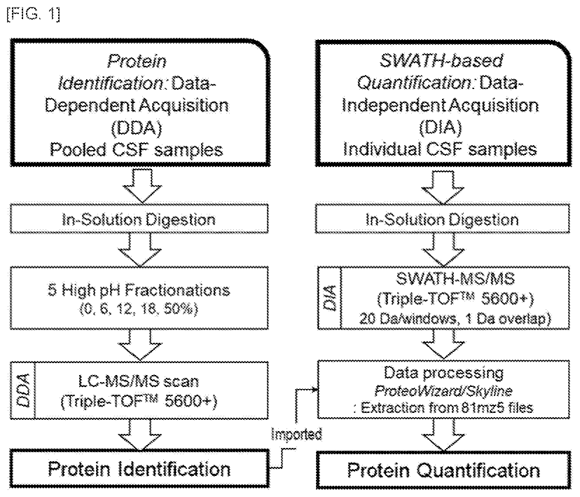

As shown in FIG. 1, in preparation for the analysis, 100 μL of two pooled cerebrospinal fluid (CSF) samples were used. The proteins were denatured and digested into peptides through pretreatment. The whole sample of digested peptides was identified as individual fractions, and spectral libraries for quantification were obtained. Filtering was performed at a 1% FDR condition, and then a total of 301 identified proteins were obtained (sequence-specific peptides: a total of 2,691; sequence-specific spectra: a total of 5,366). With respect to all kinds of the proteins to be detected, libraries for SWATH-MS quantitative analysis were obtained.

For the full-scale analysis of the samples, 100 μL of each of 81 cerebrospinal fluid (CSF) samples was obtained and subjected to treatment in the same manner as in the above preparation, and proteomic analysis was performed using Triple-TOF™ 5600+ (AB Sciex, Concord, Canada). Proteins were quantified using Eksigent NanoLC-2D and nanoFlex cHiPLC systems (0.075 mm×75 μm column) connected to the machine. SWATH-MS analysis was performed using Triple-Tof 5600+ with 20 Da/mass windows in a looped product ion mode. Each SWATH window exhibited a 1 Da overlap in the range of 400 Da to 1000 Da (e.g., Experiment 1: 400 Da to 420 Da, Experiment 2: 419 Da to 440 Da, subsequent Experiment 31: specified from 979 Da to 1000 Da). With respect to the obtained values, spectra of proteins were searched using ProteinPilot™, and the corresponding proteins and quantitative values were obtained using ProteoWizard and Skyline software. Finally, individual library spectra and SWATH spectra were examined to finally demonstrate the types and quantitative values of the proteins.

### <Example 2> Selection of Nine Kinds of Proteins (NTM, PAM, PTPRN2, LY6H, VGF, YWHAZ, CHGA, CHGB, and OPCML) as Diagnostic Biomarkers for Alzheimer's Dementia

All samples of the cerebrospinal fluids from 42 patients with Alzheimer's dementia and 39 normal control groups of the same age were subjected to SWATH-MS analysis. Through a preliminary experiment using two sample solutions (about six cerebrospinal fluid samples were combined to prepare one sample solution), the types of proteins which may be accurately detected in human cerebrospinal fluid and data processing methods were identified in advance by the SWATH-MS method. Thereafter, as a main experiment, SWATH-MS proteomic analysis was individually performed for all 81 samples to confirm that 274 types of proteins were commonly detected in all samples, and their expression levels were obtained.

SWATH-MS analysis and data processing were performed under the same conditions for all samples. The expression levels of 274 proteins may be obtained as the area under the curve (AUC) of the corresponding protein peak in the spectrum of proteomic analysis. This value is not a concept that can be expressed in a specific unit of a certain concentration, but it is possible to accurately compare concentrations of proteins analyzed at the same time, and thus the method is a method commonly used to detect protein expression levels.

In other words, the types of 274 proteins detected by the SWATH-MS method, which are described in Example 1, and the statistical analysis results of comparing those in patients with Alzheimer's dementia and normal controls are shown in Tables 3 to 10 below (p-value represents a value less than the threshold of 0.2 of the Benjamini-Hochberg false discovery rate (FDR) on an independent-samples t-test; Abbreviations: FC (log 2), log 2 fold change of AUC of controls vs. patients with Alzheimer's dementia).

AUC of the peaks corresponding to respective proteins on the spectra of the SWATH-MS analysis was compared between 42 patients with Alzheimer's dementia and 39 normal controls, and as a result, as shown in Tables 3 to 10 below, out of 274 proteins, 25 proteins showed p-values of less than 0.05, determined through an independent-samples t-test, indicating significance.

TABLE 3

| | | | FC (log2) | | Rank in | |
|---|---|---|---|---|---|---|
| UniProt | | | | standard | signif- | |
| ID | Protein | p-value | Mean | deviation | icance | FDR |
| O15240 | Neurosecretory | 0.0001 | −0.43 | 0.44 | 1 | <0.05 |
| | protein VGF | | | | | |
| P31946 | 14-3-3 protein | 0.0008 | 0.31 | 0.75 | 2 | <0.05 |
| | beta/alpha | | | | | |
| P10645 | Chromogranin-A | 0.0018 | −0.55 | 0.65 | 3 | <0.05 |
| P05060 | Secretogranin-1 | 0.0021 | −0.28 | 0.47 | 4 | <0.05 |
| Q14982 | Opioid-binding | 0.0027 | −0.51 | 0.81 | 5 | <0.05 |
| | protein/cell | | | | | |
| | adhesion | | | | | |
| | molecule | | | | | |
| | | 0.0043 | −0.39 | 0.64 | 6 | >0.05 |
| | | 0.0043 | −1.87 | 1.35 | 7 | >0.05 |
| | | 0.0074 | −0.27 | 0.44 | 8 | >0.05 |
| | | 0.0107 | 0.15 | 0.37 | 9 | >0.05 |
| | | 0.0116 | −0.39 | 0.75 | 10 | >0.05 |
| | | 0.0139 | −0.24 | 0.41 | 11 | >0.05 |
| | | 0.0162 | −0.21 | 0.42 | 12 | >0.05 |
| | | 0.0185 | −0.33 | 0.62 | 13 | >0.05 |
| | | 0.0196 | −0.26 | 0.51 | 14 | >0.05 |
| | | 0.0235 | −0.35 | 0.63 | 15 | >0.05 |
| | | 0.0248 | 0.13 | 1 | 16 | >0.05 |
| | | 0.0252 | −0.38 | 0.64 | 17 | >0.05 |
| | | 0.0252 | −0.24 | 0.49 | 18 | >0.05 |
| | | 0.0265 | −0.23 | 0.47 | 19 | >0.05 |
| | | 0.0267 | −0.2 | 0.44 | 20 | >0.05 |
| | | 0.0267 | −0.26 | 0.51 | 21 | >0.05 |
| | | 0.0326 | −0.26 | 0.62 | 22 | >0.05 |
| | | 0.0326 | −0.25 | 0.45 | 23 | >0.05 |
| | | 0.0417 | −0.23 | 0.41 | 24 | >0.05 |
| | | 0.0431 | 0.13 | 0.48 | 25 | >0.05 |
| | | 0.0501 | 0.08 | 0.6 | 26 | >0.05 |
| | | 0.0526 | 0.12 | 0.61 | 27 | >0.05 |
| | | 0.0541 | −0.27 | 0.71 | 28 | >0.05 |
| | | 0.0593 | −0.24 | 0.43 | 29 | >0.05 |
| | | 0.06 | 0.1 | 0.51 | 30 | >0.05 |
| | | 0.0662 | −0.29 | 0.61 | 31 | >0.05 |
| | | 0.0711 | 0.12 | 0.71 | 32 | >0.05 |

TABLE 4

| | | | | |
|---|---|---|---|---|
| 0.0731 | −0.16 | 0.34 | 33 | >0.05 |
| 0.0751 | −0.4 | 0.52 | 34 | >0.05 |
| 0.078 | 0.07 | 0.7 | 35 | >0.05 |
| 0.08 | −0.32 | 0.7 | 36 | >0.05 |
| 0.0944 | −0.33 | 0.69 | 37 | >0.05 |
| 0.0945 | 0.09 | 0.51 | 38 | >0.05 |
| 0.1018 | 0.08 | 0.48 | 39 | >0.05 |
| 0.102 | −0.21 | 0.44 | 40 | >0.05 |
| 0.1034 | −0.25 | 0.52 | 41 | >0.05 |
| 0.1039 | −0.18 | 0.44 | 42 | >0.05 |
| 0.1042 | −0.16 | 1.4 | 43 | >0.05 |
| 0.1064 | −0.23 | 0.56 | 44 | >0.05 |
| 0.1073 | 0.1 | 0.48 | 45 | >0.05 |
| 0.1079 | −0.22 | 0.59 | 46 | >0.05 |
| 0.1122 | 0.1 | 0.6 | 47 | >0.05 |
| 0.1178 | −0.49 | 0.9 | 48 | >0.05 |
| 0.1196 | −0.29 | 0.65 | 49 | >0.05 |
| 0.1218 | −0.2 | 0.46 | 50 | >0.05 |
| 0.1223 | −0.16 | 0.42 | 51 | >0.05 |
| 0.1282 | 0.05 | 0.59 | 52 | >0.05 |
| 0.1283 | −0.17 | 0.44 | 53 | >0.05 |
| 0.1298 | 0.06 | 0.65 | 54 | >0.05 |
| 0.1307 | −0.18 | 0.43 | 55 | >0.05 |
| 0.1332 | 0.07 | 0.58 | 56 | >0.05 |
| 0.1421 | −0.52 | 0.97 | 57 | >0.05 |
| 0.1425 | −0.58 | 1.06 | 58 | >0.05 |
| 0.1428 | −0.25 | 0.62 | 59 | >0.05 |
| 0.1492 | 0.06 | 0.39 | 60 | >0.05 |
| 0.1492 | 0.06 | 0.47 | 61 | >0.05 |
| 0.1524 | 0.06 | 0.38 | 62 | >0.05 |
| 0.1526 | −0.29 | 0.63 | 63 | >0.05 |
| 0.153 | 0.05 | 0.66 | 64 | >0.05 |
| 0.1548 | −0.36 | 0.79 | 65 | >0.05 |
| 0.1553 | −0.13 | 1.24 | 66 | >0.05 |
| 0.1618 | −0.43 | 0.87 | 67 | >0.05 |
| 0.1702 | 0.06 | 0.47 | 68 | >0.05 |
| 0.1736 | 0.06 | 0.54 | 69 | >0.05 |
| 0.174 | −0.23 | 0.64 | 70 | >0.05 |

TABLE 5

| | | | | |
|---|---|---|---|---|
| 0.174 | −0.02 | 0.87 | 71 | >0.05 |
| 0.1777 | 0.05 | 0.63 | 72 | >0.05 |
| 0.1784 | 0.05 | 0.54 | 73 | >0.05 |
| 0.1804 | −0.17 | 0.43 | 74 | >0.05 |
| 0.1811 | 0.05 | 0.55 | 75 | >0.05 |
| 0.1861 | −0.33 | 0.75 | 76 | >0.05 |
| 0.1877 | −0.18 | 0.47 | 77 | >0.05 |
| 0.1895 | −0.23 | 0.55 | 78 | >0.05 |
| 0.1902 | −0.07 | 1 | 79 | >0.05 |
| 0.1956 | 0.04 | 0.54 | 80 | >0.05 |
| 0.1982 | −0.5 | 0.85 | 81 | >0.05 |
| 0.2022 | 0.05 | 0.43 | 82 | >0.05 |
| 0.2025 | −0.51 | 0.88 | 83 | >0.05 |
| 0.2032 | 0.04 | 0.49 | 84 | >0.05 |
| 0.206 | 0.04 | 0.53 | 85 | >0.05 |
| 0.213 | −0.25 | 0.75 | 86 | >0.05 |
| 0.2135 | 0.05 | 0.38 | 87 | >0.05 |
| 0.2149 | 0.02 | 0.64 | 88 | >0.05 |
| 0.2158 | −0.34 | 0.95 | 89 | >0.05 |
| 0.2202 | −0.22 | 0.56 | 90 | >0.05 |
| 0.2205 | −0.22 | 0.59 | 91 | >0.05 |
| 0.2253 | −0.01 | 0.79 | 92 | >0.05 |
| 0.2263 | 0.05 | 0.42 | 93 | >0.05 |
| 0.2283 | 0.03 | 0.5 | 94 | >0.05 |
| 0.2314 | −0.26 | 0.82 | 95 | >0.05 |
| 0.2327 | 0.01 | 0.61 | 96 | >0.05 |
| 0.233 | 0.03 | 0.54 | 97 | >0.05 |
| 0.2418 | −0.4 | 0.82 | 98 | >0.05 |
| 0.2431 | −0.16 | 0.41 | 99 | >0.05 |
| 0.2477 | 0.02 | 0.55 | 100 | >0.05 |
| 0.2495 | 0.03 | 0.49 | 101 | >0.05 |
| 0.2503 | −0.13 | 0.4 | 102 | >0.05 |
| 0.2519 | −0.06 | 0.96 | 103 | >0.05 |
| 0.252 | 0.04 | 0.43 | 104 | >0.05 |
| 0.2557 | 0.04 | 0.45 | 105 | >0.05 |
| 0.2577 | 0.03 | 0.53 | 106 | >0.05 |
| 0.2589 | 0.01 | 0.67 | 107 | >0.05 |
| 0.2602 | 0 | 0.71 | 108 | >0.05 |

TABLE 6

| | | | | |
|---|---|---|---|---|
| 0.2643 | −0.14 | 0.43 | 109 | >0.05 |
| 0.2658 | 0.03 | 0.58 | 110 | >0.05 |
| 0.2699 | −0.19 | 0.58 | 111 | >0.05 |
| 0.2701 | 0 | 0.63 | 112 | >0.05 |
| 0.2719 | 0.05 | 0.52 | 113 | >0.05 |
| 0.2747 | 0.04 | 0.32 | 114 | >0.05 |
| 0.2774 | −0.19 | 0.56 | 115 | >0.05 |
| 0.2858 | 0 | 0.64 | 116 | >0.05 |
| 0.2878 | −0.16 | 0.51 | 117 | >0.05 |
| 0.2919 | −0.2 | 0.56 | 118 | >0.05 |
| 0.2921 | −0.26 | 0.83 | 119 | >0.05 |
| 0.2935 | −0.15 | 0.42 | 120 | >0.05 |
| 0.2977 | −0.15 | 0.46 | 121 | >0.05 |
| 0.2994 | −0.09 | 0.96 | 122 | >0.05 |
| 0.2995 | −0.22 | 0.6 | 123 | >0.05 |
| 0.2999 | 0.02 | 0.53 | 124 | >0.05 |
| 0.3004 | 0 | 0.59 | 125 | >0.05 |
| 0.3008 | −0.06 | 0.88 | 126 | >0.05 |
| 0.314 | 0.02 | 0.49 | 127 | >0.05 |
| 0.3166 | 0.02 | 0.32 | 128 | >0.05 |
| 0.3173 | 0.01 | 0.51 | 129 | >0.05 |
| 0.3178 | −0.07 | 0.87 | 130 | >0.05 |
| 0.3207 | −0.16 | 0.56 | 131 | >0.05 |
| 0.3316 | −0.3 | 0.76 | 132 | >0.05 |
| 0.3347 | −0.03 | 0.77 | 133 | >0.05 |
| 0.3363 | −0.07 | 0.84 | 134 | >0.05 |
| 0.3367 | 0.01 | 0.58 | 135 | >0.05 |
| 0.3368 | 0 | 0.58 | 136 | >0.05 |
| 0.3409 | −0.07 | 0.87 | 137 | >0.05 |
| 0.3454 | −0.16 | 0.51 | 138 | >0.05 |
| 0.3482 | −0.12 | 0.41 | 139 | >0.05 |
| 0.3497 | −0.16 | 0.54 | 140 | >0.05 |
| 0.3518 | −0.06 | 0.86 | 141 | >0.05 |
| 0.3523 | −0.08 | 0.31 | 142 | >0.05 |
| 0.3581 | −0.02 | 0.64 | 143 | >0.05 |
| 0.3606 | −0.09 | 0.34 | 144 | >0.05 |
| 0.3739 | −0.01 | 0.6 | 145 | >0.05 |
| 0.3784 | −0.06 | 0.76 | 146 | >0.05 |

TABLE 7

| | | | | |
|---|---|---|---|---|
| 0.3828 | −0.2 | 0.64 | 147 | >0.05 |
| 0.3861 | 0.02 | 0.36 | 148 | >0.05 |
| 0.392 | −0.19 | 0.62 | 149 | >0.05 |
| 0.3968 | −0.01 | 0.57 | 150 | >0.05 |
| 0.4016 | 0.01 | 0.47 | 151 | >0.05 |
| 0.4031 | 0.01 | 0.47 | 152 | >0.05 |
| 0.404 | −0.03 | 0.63 | 153 | >0.05 |
| 0.4193 | 0 | 0.54 | 154 | >0.05 |
| 0.4195 | −0.01 | 0.56 | 155 | >0.05 |
| 0.4198 | −0.04 | 0.67 | 156 | >0.05 |
| 0.42 | −0.4 | 0.85 | 157 | >0.05 |
| 0.423 | −0.51 | 1.06 | 158 | >0.05 |
| 0.4371 | −0.18 | 0.56 | 159 | >0.05 |
| 0.4377 | −0.13 | 0.49 | 160 | >0.05 |
| 0.4415 | −0.42 | 0.9 | 161 | >0.05 |
| 0.4442 | −0.45 | 0.98 | 162 | >0.05 |
| 0.4469 | −0.03 | 0.62 | 163 | >0.05 |
| 0.4478 | −0.06 | 0.71 | 164 | >0.05 |
| 0.4492 | −0.1 | 0.39 | 165 | >0.05 |
| 0.4496 | −0.06 | 0.72 | 166 | >0.05 |
| 0.4499 | −0.11 | 0.41 | 167 | >0.05 |
| 0.4522 | −0.02 | 0.65 | 168 | >0.05 |
| 0.4603 | −0.29 | 0.82 | 169 | >0.05 |
| 0.4662 | −0.05 | 0.66 | 170 | >0.05 |
| 0.4771 | −0.18 | 1 | 171 | >0.05 |
| 0.4785 | −0.13 | 0.48 | 172 | >0.05 |
| 0.4786 | 0 | 0.44 | 173 | >0.05 |
| 0.4869 | 0 | 0.39 | 174 | >0.05 |
| 0.4905 | −0.31 | 0.97 | 175 | >0.05 |
| 0.4921 | −0.39 | 0.97 | 176 | >0.05 |
| 0.4938 | 0 | 0.42 | 177 | >0.05 |
| 0.4947 | −0.03 | 0.61 | 178 | >0.05 |
| 0.4961 | −0.03 | 0.61 | 179 | >0.05 |
| 0.5002 | −0.1 | 0.4 | 180 | >0.05 |
| 0.5009 | −0.17 | 0.53 | 181 | >0.05 |
| 0.505 | −0.04 | 0.68 | 182 | >0.05 |
| 0.5126 | −0.02 | 0.53 | 183 | >0.05 |
| 0.5145 | −0.23 | 0.73 | 184 | >0.05 |

TABLE 8

| | | | | |
|---|---|---|---|---|
| 0.5179 | −0.24 | 0.73 | 185 | >0.05 |
| 0.5199 | −0.01 | 0.49 | 186 | >0.05 |
| 0.5235 | −0.1 | 0.4 | 187 | >0.05 |
| 0.5283 | −0.01 | 0.43 | 188 | >0.05 |
| 0.5293 | −0.18 | 0.69 | 189 | >0.05 |
| 0.539 | −0.01 | 0.43 | 190 | >0.05 |
| 0.5535 | −0.04 | 0.57 | 191 | >0.05 |
| 0.5625 | −0.21 | 0.74 | 192 | >0.05 |
| 0.5654 | −0.07 | 0.66 | 193 | >0.05 |
| 0.58 | −0.01 | 0.43 | 194 | >0.05 |
| 0.5845 | −0.04 | 0.54 | 195 | >0.05 |
| 0.5895 | −0.03 | 0.52 | 196 | >0.05 |
| 0.596 | −0.01 | 0.41 | 197 | >0.05 |
| 0.5967 | −0.05 | 0.58 | 198 | >0.05 |
| 0.6027 | −0.1 | 0.45 | 199 | >0.05 |
| 0.6086 | −0.17 | 0.61 | 200 | >0.05 |
| 0.609 | −0.02 | 0.5 | 201 | >0.05 |
| 0.6111 | −0.12 | 0.5 | 202 | >0.05 |
| 0.6165 | −0.19 | 0.66 | 203 | >0.05 |
| 0.6228 | −0.14 | 0.58 | 204 | >0.05 |
| 0.6376 | −0.01 | 0.46 | 205 | >0.05 |
| 0.6402 | −0.11 | 0.49 | 206 | >0.05 |
| 0.6442 | −0.09 | 0.74 | 207 | >0.05 |
| 0.6456 | −0.03 | 0.45 | 208 | >0.05 |
| 0.6474 | 0 | 0.33 | 209 | >0.05 |
| 0.6594 | −0.16 | 0.6 | 210 | >0.05 |
| 0.6599 | −0.18 | 1.04 | 211 | >0.05 |
| 0.6624 | −0.05 | 0.57 | 212 | >0.05 |
| 0.6784 | −0.04 | 0.48 | 213 | >0.05 |
| 0.6818 | −0.48 | 1.17 | 214 | >0.05 |
| 0.6935 | −0.49 | 1.46 | 215 | >0.05 |
| 0.6981 | −0.08 | 0.63 | 216 | >0.05 |
| 0.6987 | −0.13 | 0.59 | 217 | >0.05 |
| 0.6987 | −0.06 | 0.55 | 218 | >0.05 |
| 0.7075 | −0.12 | 0.83 | 219 | >0.05 |
| 0.7077 | −0.09 | 0.66 | 220 | >0.05 |
| 0.7085 | −0.02 | 0.39 | 221 | >0.05 |
| 0.7244 | −0.07 | 0.39 | 222 | >0.05 |

TABLE 9

| | | | | |
|---|---|---|---|---|
| 0.7367 | −0.08 | 0.61 | 223 | >0.05 |
| 0.7447 | −0.1 | 0.47 | 224 | >0.05 |
| 0.7451 | −0.06 | 0.33 | 225 | >0.05 |
| 0.7556 | −0.12 | 0.74 | 226 | >0.05 |
| 0.7663 | −0.02 | 0.34 | 227 | >0.05 |
| 0.7688 | −0.1 | 0.68 | 228 | >0.05 |
| 0.775 | −0.21 | 1.04 | 229 | >0.05 |
| 0.7803 | −0.06 | 0.54 | 230 | >0.05 |
| 0.7835 | −0.05 | 0.54 | 231 | >0.05 |
| 0.7845 | −0.09 | 0.48 | 232 | >0.05 |
| 0.7959 | −0.24 | 0.87 | 233 | >0.05 |
| 0.8047 | −0.09 | 0.6 | 234 | >0.05 |
| 0.8135 | −0.12 | 0.55 | 235 | >0.05 |
| 0.8142 | −0.04 | 0.42 | 236 | >0.05 |
| 0.8182 | −0.08 | 0.6 | 237 | >0.05 |
| 0.8192 | −0.02 | 0.35 | 238 | >0.05 |
| 0.8238 | −0.04 | 0.41 | 239 | >0.05 |
| 0.833 | −0.33 | 1.18 | 240 | >0.05 |
| 0.8406 | −0.09 | 0.54 | 241 | >0.05 |
| 0.8417 | −0.12 | 0.58 | 242 | >0.05 |
| 0.848 | −0.09 | 0.53 | 243 | >0.05 |
| 0.8523 | −0.17 | 0.84 | 244 | >0.05 |
| 0.8569 | −0.1 | 0.61 | 245 | >0.05 |
| 0.8615 | −0.03 | 0.37 | 246 | >0.05 |
| 0.8527 | −0.08 | 0.46 | 247 | >0.05 |
| 0.874 | −0.04 | 0.42 | 248 | >0.05 |
| 0.888 | −0.22 | 0.93 | 249 | >0.05 |
| 0.8884 | −0.14 | 0.67 | 250 | >0.05 |
| 0.8897 | −0.06 | 0.39 | 251 | >0.05 |
| 0.8925 | −0.12 | 0.55 | 252 | >0.05 |
| 0.8957 | −0.04 | 0.31 | 253 | >0.05 |
| 0.8995 | −0.06 | 0.42 | 254 | >0.05 |
| 0.9037 | −0.07 | 0.43 | 255 | >0.05 |
| 0.9097 | −0.14 | 0.71 | 256 | >0.05 |
| 0.9151 | −0.04 | 0.36 | 257 | >0.05 |
| 0.9179 | −0.23 | 0.9 | 258 | >0.05 |
| 0.918 | −0.18 | 0.79 | 259 | >0.05 |
| 0.9308 | −0.1 | 0.53 | 260 | >0.05 |

TABLE 10

| | | | | |
|---|---|---|---|---|
| 0.9312 | −0.16 | 0.75 | 261 | >0.05 |
| 0.9315 | −0.69 | 1.29 | 262 | >0.05 |
| 0.9401 | −0.06 | 0.4 | 263 | >0.05 |
| 0.9401 | −0.02 | 0.26 | 264 | >0.05 |
| 0.9427 | −0.06 | 0.46 | 265 | >0.05 |
| 0.9572 | −0.03 | 0.31 | 266 | >0.05 |
| 0.9576 | −0.2 | 0.78 | 267 | >0.05 |
| 0.958 | −0.15 | 0.66 | 268 | >0.05 |
| 0.96 | −0.04 | 0.36 | 269 | >0.05 |
| 0.9654 | −0.05 | 0.41 | 270 | >0.05 |
| 0.9664 | −0.17 | 0.76 | 271 | >0.05 |
| 0.9664 | −0.04 | 0.42 | 272 | >0.05 |
| 0.9709 | −0.11 | 0.59 | 273 | >0.05 |
| 0.9968 | −0.11 | 0.57 | 274 | >0.05 |

However, since the samples of 274 proteins were compared at the same time, the p-values were reevaluated using the Benjamini-Hochberg false discovery rate (FDR), which is a statistical technique to reduce statistical errors caused by repeated comparisons. In other words, only the proteins with FDR <0.2 were finally selected as those with significant difference. Accordingly, a total of nine proteins including neurotrimin (NTM), peptidylglycine-α-amidating monooxygenase (PAM), receptor-type tyrosine-protein phosphatase N2 (PTPRN2), lymphocyte antigen 6H (LY6H), and neurosecretory protein VGF, 14-3-3 protein ζ/δ (YWHAZ), chromogranin-A (CHGA), secretogranin-1 (CHGB), and opioid-binding protein/cell adhesion molecule (OPCML) with FDR <0.05 were selected. The nine kinds of proteins showing significant differences in patients with Alzheimer's dementia and statistical values thereof are shown in Table 11 below.

TABLE 11

| | | | AUC | | | |
|---|---|---|---|---|---|---|
| UniProt ID | Protein | Gene | Control | AD | p-Value | FC (log2) |
| O15240 | Neurosecretory protein | VGF | 23478.5 ± 6236.6 | 18243.9 ± 5387.3 | 0.0001** | −0.4 ± 0.4 |
| | VGF | | | | | |
| P63104 | 14-3-3 protein ζ/δ | YWHAZ | 1219.8 ± 502.4 | 1676.7 ± 712.5 | 0.0012** | 0.3 ± 0.75 |
| P10645 | Chromogranin-A | CHGA | 7950.8 ± 3236.5 | 5909.1 ± 2302.6 | 0.0018** | −0.6 ± 0.6 |
| P05060 | Secretogranin-1 | CHGB | 46505.7 ± 9216.4 | 39804.8 ± 9753.9 | 0.0021** | −0.3 ± 0.5 |
| Q14982 | Opioid-binding | OPCML | 7684.5 ± 2262.7 | 6039.4 ± 2498.3 | 0.0027** | −0.5 ± 0.8 |
| | protein/cell adhesion | | | | | |
| | molecule | | | | | |
| Q9P121 | Neurotrimin | NTM | 13841.1 ± 2094.8 | 12390.1 ± 3084.6 | 0.0162* | −0.2 ± 0.4 |
| P19021 | Peptidylglycine-α- | PAM | 2776.4 ± 642.9 | 2382.8 ± 823.6 | 0.0185* | −0.3 ± 0.6 |
| | amidating | | | | | |
| | monooxygenase | | | | | |
| O94772 | Lymphocyte antigen 6H | LY6H | 6568.1 ± 1798.8 | 5567.3 ± 2098.1 | 0.0235* | −0.4 ± 0.6 |
| Q92932 | Receptor-type tyrosine- | PTPRN2 | 3469.8 ± 1047.5 | 2914.9 ± 1135.2 | 0.0252* | −0.2 ± 0.5 |
| | protein phosphatase N2 | | | | | |

**FDR < 0.05
*FDR < 0.2

AD, Alzheimer's dementia; AUC, area under the curve of spectral peak of corresponding protein in SWATH-MS analysis; FC (log 2), log 2 fold change of AUC of control vs. Alzheimer's dementia.

Among the selected nine kinds of proteins, the top five proteins were subjected to ROC curve analysis to validate their utility in the diagnosis of Alzheimer's dementia. As a result, as confirmed in Table 12 below, the AUC value was measured to be around 0.7. It was confirmed that the five kinds of proteins may also be individually used for the diagnosis of Alzheimer's dementia.

TABLE 12

| | | | | Youden | Sen | Spe |
|---|---|---|---|---|---|---|
| | AUC | 95% CI | p-Value | index J | (%) | (%) |
| Neurosecretory | 0.752 | 0.642-0.861 | <0.001 | 0.51 | 82 | 69 |
| protein VGF | | | | | | |
| (VGF) | | | | | | |
| 14-3-3 protein ζ/δ | 0.697 | 0.579-0.815 | 0.002 | 0.38 | 69 | 69 |
| (YWHAZ) | | | | | | |
| Chromogranin-A | 0.678 | 0.559-0.796 | 0.006 | 0.37 | 56 | 81 |
| (CHGA) | | | | | | |
| Secretogranin-1 | 0.705 | 0.590-0.819 | 0.002 | 0.39 | 79 | 60 |
| (CHGB) | | | | | | |
| Opioid-binding | 0.698 | 0.583-0.814 | 0.002 | 0.39 | 79 | 60 |
| protein/cell | | | | | | |
| adhesion | | | | | | |
| molecule | | | | | | |
| (OPCML) | | | | | | |

### <Example 3> Optimal Protein Combination for Diagnosis of Alzheimer's Dementia and Examination of Utility Thereof

### Example 3-1: Selection of Optimal Protein Combination (YWHAZ, CHGA, and CHGB) for Diagnosis of Alzheimer's Dementia

In order to examine whether a combination of the five proteins selected in Example 2 is more useful than individual proteins in diagnosing Alzheimer's dementia, logistic regression with backward stepwise selection was performed. As confirmed in Table 13 showing a process of deriving a regression equation, which may explain the possibility of diagnosis of Alzheimer's dementia through simultaneous measurement of three proteins (YWHAB, CHGA, and CHGB), neurosecretory protein VGF and opioid-binding protein/cell adhesion molecule (OPCML) showed a significant probability (p-value) of more than about 0.05 in any combination. A combination of three proteins of 14-3-3 protein ζ/δ (YWHAZ), chromogranin-A (CHGA), and secretogranin-1 (CHGB), excluding the two proteins, showed the most significant probability, and thus the combination of the three proteins was selected as an optimal protein combination for the diagnosis of Alzheimer's dementia.

TABLE 13

| | | | | | | | | 95.0% C.I. for | |
|---|---|---|---|---|---|---|---|---|---|
| | | | | | | | | EXP(B) | |
| Step | | B | S.E. | Wald | df | Sig. | Exp(B) | Lower | Upper |
| Step 1^{a} | VGF | −2.135 | 2.076 | 1.058 | 1 | .304 | .118 | .002 | 6.911 |
| | YWHAZ | 3.487 | .829 | 17.694 | 1 | .000 | 32.674 | 6.437 | 165.860 |
| | CHGA | −2.611 | 1.362 | 3.675 | 1 | .055 | .073 | .005 | 1.060 |
| | CHGB | −2.719 | 2.626 | 1.073 | 1 | .300 | .066 | .000 | 11.325 |
| | OPCML | −1.063 | 1.296 | .672 | 1 | .412 | .345 | .027 | 4.384 |
| | Constant | 3.539 | 1.518 | 5.440 | 1 | .020 | 34.443 | — | — |
| Step 2^{a} | VGF | −2.182 | 2.092 | 1.088 | 1 | .297 | .113 | .002 | 6.806 |
| | YWHAZ | 3.504 | .835 | 17.612 | 1 | .000 | 33.242 | 6.472 | 170.747 |
| | CHGA | −2.638 | 1.352 | 3.807 | 1 | .051 | .071 | .005 | 1.012 |
| | CHGB | −3.728 | 2.424 | 2.365 | 1 | .124 | .024 | .000 | 2.782 |
| | Constant | 3.587 | 1.517 | 5.592 | 1 | .018 | 36.119 | — | — |
| Step 3^{a} | YWHAZ | 3.530 | .837 | 17.800 | 1 | .000 | 34.114 | 6.619 | 175.822 |
| | CHGA | −3.384 | 1.161 | 8.502 | 1 | .004 | .034 | .003 | .330 |
| | CHGB | −5.222 | 2.042 | 6.540 | 1 | .011 | .005 | .000 | .295 |
| | Constant | 3.639 | 1.520 | 5.734 | 1 | .017 | 38.038 | — | — |

^{a}variables entered in each step: Step 1: VGF, YWHAZ, CHGA, CHGB, OPCML; Step 2: VGF, YWHAZ, CHGA, CHGB; Step 3: YWHAZ, CHGA, CHGB.

### Example 3-2: Examination of Utility of Three Selected Proteins (YWHAZ, CHGA, and CHGB) for Diagnosis of Alzheimer's Dementia

A regression equation was obtained to explain the diagnosis of Alzheimer's dementia through simultaneous measurement of the three selected proteins. Regression coefficients and regression constant values were obtained, and finally, a regression equation of 3.639+3.530×YWHAZ(1433Z)−3.384×CHGA−5.222×CHGB was obtained. With the regression equation including all expression levels of the three proteins, equation values were obtained from all samples, and then ROC analysis for the diagnosis of Alzheimer's dementia was performed with the values. As a result, as shown in Table 14, a high AUC value of 0.889 was observed. In addition, through coordinate analysis of the ROC graph, a cut-off value for diagnosis was obtained from the coordinate point where the Youden index J value was highest. When based on this, the value of the regression equation had sensitivity (Sen) of 83% and specificity (Spe) of 80%, indicating that diagnosis of Alzheimer's dementia is possible.

TABLE 14

| | | | | Youden | Sen | Spe |
|---|---|---|---|---|---|---|
| | AUC | 95% C.I. | p-Value | index J | (%) | (%) |
| Regression | 0.889 | 0.819-0.959 | <0.001 | 0.63 | 83 | 80 |
| Equation | | | | | | |

In other words, as shown in the above results and FIG. 2, the combination of the three proteins exhibited a much higher ROC curve and regression equation value than the individual proteins. Thus, it was confirmed that Alzheimer's dementia may be diagnosed with high accuracy and sensitivity through simultaneous measurement of the expression levels of the three proteins (YWHAZ(1433Z), CHGA, and CHGB) as compared with measurement of the individual proteins.

### <Example 4> Mini-Mental State Examination (MMSE)

Mini-mental state examination (MMSE), which is a tool for screening dementia, and which is capable of evaluating cognitive impairment due to Alzheimer's disease, was performed to analyze a correlation with the markers.

More specifically, the correlation between the mini-mental state examination (MMSE) scores and the log 2 fold change in the expression levels of the proteins in cerebrospinal fluid was analyzed through Spearman's rank correlation analysis. The MMSE score decreases as cognitive impairment becomes severe.

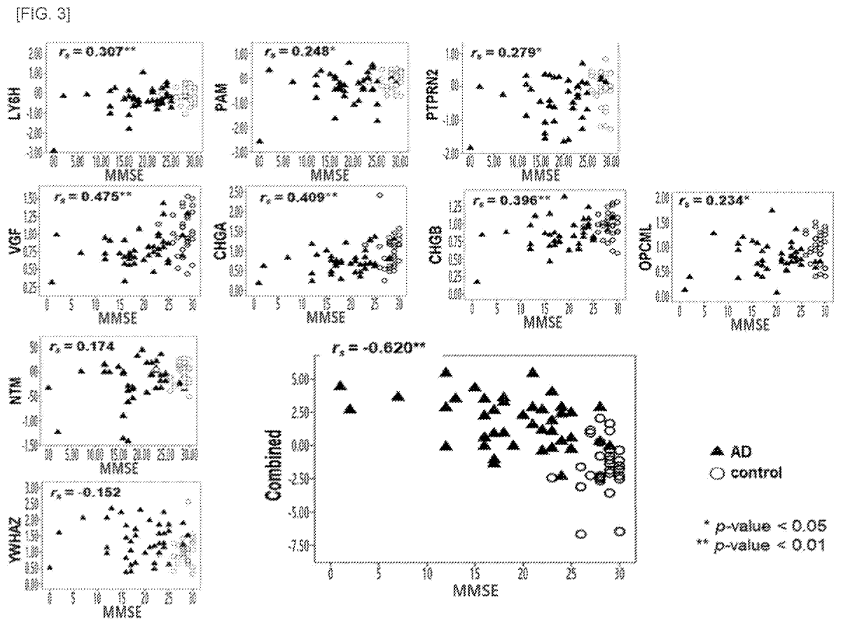

As a result, as shown in FIG. 3, a correlation coefficient of 0.307 (p-value, 0.009) was observed between the LY6H protein level in the cerebrospinal fluid and the MMSE score, a correlation coefficient of 0.248 (p-value, 0.037) in PAM, a correlation coefficient of 0.279 (p-value, 0.019) in PTPRN2, a correlation coefficient of 0.475 (p-value, <0.0001) in VGF, a correlation coefficient of 0.409 (p-value, 0.0004) in CHGA, a correlation coefficient of 0.396 (p-value, 0.0006) in CHGB, and a correlation coefficient of 0.234 (p-value, 0.0498) in OPCML, indicating that all showed a significant correlation, i.e., cognitive function declined as each protein level decreased. NTM showed a correlation coefficient of 0.174 (p-value, 0.147), and YWHAZ showed a correlation coefficient of −0.152 (p-value, 0.206), indicating no correlation between changes in the protein levels and decline in the cognitive function. However, the regression equation (combined) value, which is a combination of YWHAZ, CHGA, and CHGB, showed a correlation coefficient of −0.620 (p-value, <0.0001), indicating a high level of significant correlation, in which cognitive function declined, as the value increased.

These results confirmed that the changes of the expression levels of the proteins in the cerebrospinal fluid and the severity of cognitive impairment of dementia patients have a significant correlation, and this correlation was further increased when YWHAZ, CHGA, and CHGB were combined.

### <Example 5> Clinical Dementia Rating-Sum-of-Box (CDR-SOB) Analysis

Clinical dementia rating-sum-of-box (CDR-SOB) capable of clinically evaluating the severity of dementia patients was performed to analyze the correlation with the markers.

In more detail, a correlation between the clinical dementia rating-sum-of-box (CDR-SOB) scores and log 2 fold change levels of the proteins in the cerebrospinal fluid was demonstrated through Spearman's rank correlation analysis. CDR-SOB means the sum of values, obtained by rating five items of memory, orientation, judgment/problem solving, community affairs, home and hobbies, and hygiene and dressing with 0, 0.5, 1, 2, 3, 4, or 5 points, respectively. As the dementia becomes more severe, the score becomes higher.

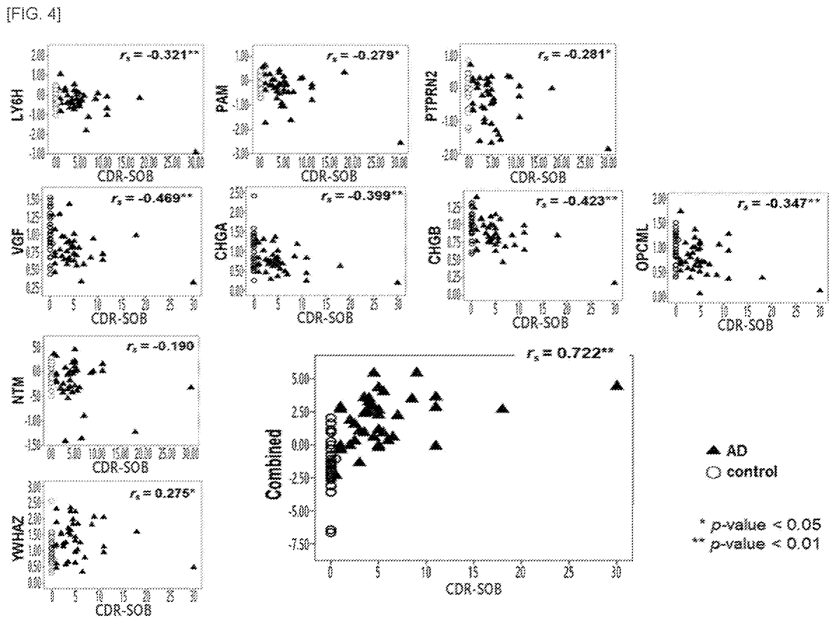

As a result, as shown in FIG. 4, a correlation coefficient of −0.321 (p-value, 0.006) was observed between the LY6H protein level in the cerebrospinal fluid and the CDR-SOB score, a correlation coefficient of −0.279 (p-value, 0.017) in PAM, a correlation coefficient of −0.281 (p-value, 0.016) in PTPRN2, a correlation coefficient of −0.469 (p-value, <0.0001) in VGF, a correlation coefficient of −0.399 (p-value, 0.0005) in CHGA, a correlation coefficient of −0.423 (p-value, 0.0002) in CHGB, and a correlation coefficient of −0.347 (p-value, 0.0026) in OPCML, indicating that all showed a significant correlation, i.e., the severity of dementia increased as each protein level decreased. NTM showed a correlation coefficient of −0.190 (p-value, 0.107), indicating no correlation between changes in the protein levels and the severity of dementia. YWHAZ showed a correlation coefficient of 0.275 (p-value, 0.0185), indicating a significant correlation, i.e., the severity of dementia increased as the protein level increased. When the YWHAZ level was combined with the CHGA and CHGB levels through regression equation (combined), the correlation coefficient was 0.722 (p-value, <0.0001), indicating that the significant correlation was further enhanced, i.e., the severity of dementia increased as the protein level increased.

These results confirmed that the changes of the expression levels of the proteins in the cerebrospinal fluid and the severity of dementia patients have a significant correlation, and this correlation was further increased when YWHAZ, CHGA, and CHGB were combined.

Taken together, the present disclosure confirmed that among the 274 proteins commonly detected in patients with Alzheimer's dementia, five proteins (VGF, YWHAZ, CHGA, CHGB, and OPCML) showed significantly different expression levels as compared with the normal control group. Moreover, it was confirmed that a combination of the three proteins (YWHAZ, CHGA, and CHGB) showed a remarkably high sensitivity and specificity when used in diagnosing Alzheimer's dementia, as compared with the five individual proteins. Through this, it was confirmed that useful information for diagnosis or prognosis of Alzheimer's dementia may be obtained through simultaneous measurement of the expression levels of the three proteins. It was also confirmed that changes in the expression patterns of the proteins showed a significant correlation with the degree of cognitive impairment and the severity of dementia in dementia patients. Finally, a total of nine proteins including NTM, PAM, PTPRN2, LY6H, VGF, YWHAZ, CHGA, CHGB, and combinations thereof may be useful biomarkers for diagnosing Alzheimer's dementia or predicting severity thereof.

In the present disclosure, detailed descriptions of the contents that may be sufficiently recognized and inferred by those skilled in the art have been omitted. In addition to the specific embodiments described herein, more various modifications are possible within a range that does not change the technical spirit or essential characteristics thereof. Accordingly, the present disclosure may be implemented in a manner different from those specifically described and illustrated in the present specification, which may be understood by one of ordinary skill in the art to which the present disclosure belongs.

### Effect of the Invention

A composition for diagnosing Alzheimer's dementia of the present disclosure may be used to diagnose Alzheimer's dementia with high sensitivity and specificity by measuring expression levels of mRNAs of seven kinds of genes (neurotrimin (NTM), peptidylglycine-α-amidating monooxygenase (PAM), receptor-type tyrosine-protein phosphatase N2 (PTPRN2), opioid-binding protein/cell adhesion molecule (OPCML), 14-3-3 protein ζ/δ (YWHAZ), chromogranin-A (CHGA), and secretogranin-1 (CHGB)) or expression levels of proteins expressed therefrom, and then comparing the levels with those of a normal control group.

1-14. (canceled)

15. A method of providing information for diagnosis of Alzheimer's dementia, the method comprising the steps of:
(a) measuring levels of mRNAs of one or more genes selected from the group consisting of neurotrimin (NTM), peptidylglycine-α-amidating monooxygenase (PAM), receptor-type tyrosine-protein phosphatase N2 (PTPRN2), opioid-binding protein/cell adhesion molecule (OPCML), 14-3-3 protein ζ/δ (YWHAZ), chromogranin-A (CHGA), and secretogranin-1 (CHGB), or levels of proteins expressed therefrom in a sample isolated from an individual suspected of having Alzheimer's dementia; and
(b) comparing the measured levels with those measured in a sample isolated from a normal individual.

16. The method of claim 15, wherein the mRNA levels of the genes are measured by reverse transcriptase polymerase chain reaction (RT-PCR), competitive reverse transcriptase polymerase chain reaction (competitive RT-PCR), real-time quantitative RT-PCR, an RNase protection method, Northern blotting, or a DNA chip technology assay.

17. The method of claim 15, wherein the levels of the proteins are measured by any one selected from the group consisting of Western blotting, enzyme-linked immunosorbent assay (ELISA), radioimmunoassay (RIA), radioimmunodiffusion, Ouchterlony immunodiffusion, rocket immunoelectrophoresis, immunohistochemical staining, immunoprecipitation assay, complement fixation assay, immunofluorescence, immunochromatography, fluorescence-activated cell sorter analysis (FACS), and a protein chip technology assay.

18. The method of claim 15, wherein the sample is a tissue, a cell, whole blood, serum, plasma, saliva, sputum, cerebrospinal fluid, or urine isolated from the individual.

19. The method of claim 15, wherein the gene is a combination of YWHAB, CHGA, and CHGB.

20. The method of claim 19, wherein the gene further includes any one or more genes selected from the group consisting of NTM, PAM, lymphocyte antigen 6 family member H (LY6H), PTPRN2, OPCML, and VGF (neurosecretory protein VGF).

21. The method of any one of claim 15, wherein i) when the level of mRNA or protein of YWHAZ gene measured in the sample isolated from the individual suspected of having Alzheimer's dementia is high, or ii) when the level of mRNA or protein of NTM, PAM, PTPRN2, LY6H, VGF, CHGA, CHGB, or OPCML gene measured in the sample isolated from the individual suspected of having Alzheimer's dementia is low, as compared with the level measured in a sample isolated from a normal individual, it is determined that the risk of Alzheimer's dementia is high.
